SEQUENCE LISTING (57 sequences)

SEQ ID NO: 1 - protein, 30 aa, Homo sapiens
PGNYSFSYQLEDEPWKLCRLHQAPTARGAV

SEQ ID NO: 2 - DNA, 349 bp, Homo sapiens
caggtgcagctgcaggagtcgggcccaggactggtgaagccttcggagaccctgtccctcacctgcactgtctctggtgcctccatcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgaggtctgtgaccgctgcggacacggccgtgtattactgtgcgagagagcgactggggatcggggactactggggccaaggaaccctggtcaccgtctcctcag

SEQ ID NO: 3 - protein, 116 aa, Homo sapiens
QVQLQESGPGLVKPSETLSLTCTVSGASISSYYWSWIRQPPGKGLEWIGYIYYSGSTNYNPSLKSRVTISVDTSKNQFSLKLRSVTAADTAVYYCARERLGIGDYWGQGTLVTVSS

SEQ ID NO: 4 - DNA, 322 bp, Homo sapiens
gacatccagctgacccaatctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatacttaccctccgacgttcggccaagggaccaaggtggaaatcaaac

SEQ ID NO: 5 - protein, 107 aa, Homo sapiens
DIQLTQSPSSLSASVGDRVTITCRASQGIRNDLGWYQQKPGKAPKRLIYAASSLQSGVPSRFSGSGSGTEFTLTISSLQPEDFATYYCLQHNTYPPTFGQGTKVEIK

SEQ ID NO: 6 - DNA, 370 bp, Homo sapiens
caggtgcagctggtggagtctgggggaggcgtggtccagcctgggaggtccctgagactctcctgtgtagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagttgaggacacggctgtgtattactgtgcgagagatcacggtgggaggtacgtctacgactacggtatggacgtctggggccaagggaccacggtcaccgtctcctcag

SEQ ID NO: 7 - protein, 123 aa, Homo sapiens
QVQLVESGGGVVQPGRSLRLSCVASGFTFSSYGMHWVRQAPGKGLEWVAVISYDGSNKYYADSVKGRFTISRDNSKNTLYLQMNSLRVEDTAVYYCARDHGGRYVYDYGMDVWGQGTTVTVSS

SEQ ID NO: 8 - DNA, 322 bp, Homo sapiens
gacatccagatgacccaatctccatcttccgtgtctgcatctataggagacagagtctccatcacttgtcgggcgagtcagggtattagcagctggttagcctggtatcagcagaaaccagggaaagcccctacgctccttatctatgctgcatccactttgcaacgtggggtcccatcaaggttcagcggcagtggatctgggacagatttcactctcaccatcagcagcctgcagcctgaagattttgcaacttacttttgtcaacaggctaacagtttcccattcactttcggccctgggaccaaagtggatatcaaac

SEQ ID NO: 9 - protein, 107 aa, Homo sapiens
DIQMTQSPSSVSASIGDRVSITCRASQGISSWLAWYQQKPGKAPTLLIYAASTLQRGVPSRFSGSGSGTDFTLTISSLQPEDFATYFCQQANSFPFTFGPGTKVDIK

SEQ ID NO: 10 - DNA, 370 bp, Homo sapiens
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagttgaggacacggctgtgtattactgtgcgagagatcacggtgggaggtacgtctacgactacggtatggacgtctggggccaagggaccacggtcaccgtctcctcag

SEQ ID NO: 11 - protein, 123 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRVEDTAVYYCARDHGGRYVYDYGMDVWGQGTTVTVSS

SEQ ID NO: 12 - DNA, 322 bp, Homo sapiens
gacatccagatgacccaatctccatcttccgtgtctgcatctgtaggagacagagtctccatcacttgtcgggcgagtcagggtattagcagctggttagtctggtatcagcagaaaccagggaaagcccctgcgctcctaatctatgctgcatccagtttgcagcgtggggtcccatcaaggttcagcggcagtggatctgggacagacttcactctcaccatcagcagcctgcagcctgaagattttgcaacttacttttgtcaacaggctaacagtttcccattcactttcggccctgggaccaaagtggatatcaaac

SEQ ID NO: 13 - protein, 107 aa, Homo sapiens
DIQMTQSPSSVSASVGDRVSITCRASQGISSWLVWYQQKPGKAPALLIYAASSLQRGVPSRFSGSGSGTDFTLTISSLQPEDFATYFCQQANSFPFTFGPGTKVDIK

SEQ ID NO: 14 - DNA, 370 bp, Homo sapiens
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggtagttatatcatatgatggaagtaataaatactatgcagactccgtgaagggccgattcaccatctccagagacaattccaagaacacgctgtatctgcaaatgaacagcctgagagttgaggacacggctgtgtattactgtgcgagagatcacggtgggaggtacgtctacgactacggtatggacgtctggggccaagggaccacggtcaccgtctcctcag

SEQ ID NO: 15 - protein, 123 aa, Homo sapiens
QVQLVESGGGVVQPGRSLRLSCAASGFTFSSYGMHWVRQAPGKGLEWVVVISYDGSNKYYADSVKGRFTISRDNSKNTLYLQMNSLRVEDTAVYYCARDHGGRYVYDYGMDVWGQGTTVTVSS

SEQ ID NO: 16 - DNA, 322 bp, Homo sapiens
gacatccagatgacccaatctccatcttccgtgtctgcatctgtaggagacagagtctccatcacttgtcgggcgagtcagggtattagcagctggttagcctggtatcagcagaaaccagggaaagcccctacgctcctaatctatgctgcatccagtttgcaacgtggggtcccatcaaggttcagcggcagtggatctgggacagatttcactctcaccatcagcagcctgcagcctgaagattttgcaacttacttttgtcaacaggctaacagtttcccattcactttcggccctgggaccaaagtggatatcaaac

SEQ ID NO: 17 - protein, 107 aa, Homo sapiens
DIQMTQSPSSVSASVGDRVSITCRASQGISSWLAWYQQKPGKAPTLLIYAASSLQRGVPSRFSGSGSGTDFTLTISSLQPEDFATYFCQQANSFPFTFGPGTKVDIK

SEQ ID NO: 18 - DNA, 349 bp, Homo sapiens
caggtgcagctggtggagtcggggggaggcgtggtccagcctgggaggtccctgagactctcctgtgcagcgtctggattcaccttcagtaaatatggcatgcactgggtccgccaggctccaggcaaggggctggagtgggtggcagttttatggtatgatggaagtaataaatactatgcagactccgtgaagggccgattcaccatctccagagacaattccaagaacacgctgtatctgcaaatgaacagcctgagagccgaggacacggctgtgtattactgtgcgagaggtccgtactactttgactactggggccagggaaccctggtcaccgtctcctcag

SEQ ID NO: 19 - protein, 116 aa, Homo sapiens
QVQLVESGGGVVQPGRSLRLSCAASGFTFSKYGMHWVRQAPGKGLEWVAVLWYDGSNKYYADSVKGRFTISRDNSKNTLYLQMNSLRAEDTAVYYCARGPYYFDYWGQGTLVTVSS

SEQ ID NO: 20 - DNA, 325 bp, Homo sapiens
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactgtcaccatcagcagactggaacctgaagattttgcagtgtattactgtcagcagtatggtagttcaccgtggacgttcggccaagggaccaaggtggaaatcaaac

SEQ ID NO: 21 - protein, 108 aa, Homo sapiens
EIVLTQSPGTLSLSPGERATLSCRASQSVSSSYLAWYQQKPGQAPRLLIYGASSRATGIPDRFSGSGSGTDFTVTISRLEPEDFAVYYCQQYGSSPWTFGQGTKVEIK

SEQ ID NO: 22 - DNA, 370 bp, Homo sapiens; identical to SEQ ID NO: 10

SEQ ID NO: 23 - protein, 123 aa, Homo sapiens; identical to SEQ ID NO: 11

SEQ ID NO: 24 - DNA, 322 bp, Homo sapiens
gacatccagatgacccaatctccatcttccgtgtccgcatctgtaggagacagagtctccatcacttgtcgggcgagtcagggtattagcagctggttagcctggtatcagcagaaaccagggaaagcccctacgctcctaatctatgctgcatccagtttgcaacgtggggtcccatcaaggttcagcggcagtggatctgggacagatttcactctcaccatcagcagcctgcagcctgaagattttgcaacttacttttgtcaacaggctaacagtttcccattcactttcggccctgggaccaaagtggatatcaaac

SEQ ID NO: 25 - protein, 107 aa, Homo sapiens; identical to SEQ ID NO: 17

SEQ ID NO: 26 - DNA, 370 bp, Homo sapiens; identical to SEQ ID NO: 10

SEQ ID NO: 27 - protein, 123 aa, Homo sapiens; identical to SEQ ID NO: 11

SEQ ID NO: 28 - DNA, 322 bp, Homo sapiens
gacatccagatgacccaatctccatcttccgtgtctgcatctgtaggagacagagtctccatcacttgtcgggcgagtcagggtattagcagctggttagcctggtatcagcagaaaccagggaaagcccctaagcgcctgatctatgctgcatccagtttgcaacgtggggtcccatcaaggttcagcggcagtggatctgggacagatttcactctcaccatcagcagcctgcagcctgaagattttgcaacttacttttgtcaacaggctaacagtttcccattcactttcggccctgggaccaaagtggatatcaaac

SEQ ID NO: 29 - protein, 107 aa, Homo sapiens
DIQMTQSPSSVSASVGDRVSITCRASQGISSWLAWYQQKPGKAPKRLIYAASSLQRGVPSRFSGSGSGTDFTLTISSLQPEDFATYFCQQANSFPFTFGPGTKVDIK

SEQ ID NO: 30 - DNA, 370 bp, Homo sapiens; identical to SEQ ID NO: 10

SEQ ID NO: 31 - protein, 123 aa, Homo sapiens; identical to SEQ ID NO: 11

SEQ ID NO: 32 - DNA, 322 bp, Homo sapiens
gacatccagatgacccagtctccatcttccgtgtctacatctgtaggagacagagtctccatcacttgtcgggcgagtcagggtattggcagctggttagcctggtatcagcagaaaccagggcaagcccctacgctcctaatctatgctgcatccagtttgcaacgtggggtcccatcaagattcagcggcagtggatctgggacagatttcactctcaccatcaacagcctgcagcctgaagattttgcaacttacttttgtcaacaggctaacagtttcccattcactttcggccctgggaccaaagtggatgtcaaac

SEQ ID NO: 33 - protein, 107 aa, Homo sapiens
DIQMTQSPSSVSTSVGDRVSITCRASQGIGSWLAWYQQKPGQAPTLLIYAASSLQRGVPSRFSGSGSGTDFTLTINSLQPEDFATYFCQQANSFPFTFGPGTKVDVK

SEQ ID NO: 34 - DNA, 370 bp, Homo sapiens; identical to SEQ ID NO: 10

SEQ ID NO: 35 - protein, 123 aa, Homo sapiens; identical to SEQ ID NO: 11

SEQ ID NO: 36 - DNA, 322 bp, Homo sapiens
gacatccagatgacccagtctccatcttccgtgtctgcatctgtaggagacagagtctccatcacttgtcgggcgagtcagggtattggcagctggttagcctggtatcagcagaaaccagggcaagcccctacgctcctaatctatgctgcatccagtttgcaacgtggggtcccatcaagattcagcggcagtggatctgggacagatttcactctcaccatcaacagcctgcagcctgaagattttgcaacttacttttgtcaacaggctaacagtttcccattcactttcggccctgggaccaaagtggatgtcaaac

SEQ ID NO: 37 - protein, 107 aa, Homo sapiens
DIQMTQSPSSVSASVGDRVSITCRASQGIGSWLAWYQQKPGQAPTLLIYAASSLQRGVPSRFSGSGSGTDFTLTINSLQPEDFATYFCQQANSFPFTFGPGTKVDVK

SEQ ID NO: 38 - DNA, 349 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtttgatggaaataataaattctatgcagactccgtgaagggccgattcaccatctccagagacaattccaagaacacgctgtatctgcaaatgaacagcctgagagtcgaggacacggctgtgtattactgtgcgcgaggcgggagctactgggactactggggccagggaaccctggtcaccgtctcctcag

SEQ ID NO: 39 - protein, 116 aa, Homo sapiens
QVQLVESGGGVVQPGRSLRLSCAASGFTFSSYGMHWVRQAPGKGLEWVAVIWFDGNNKFYADSVKGRFTISRDNSKNTLYLQMNSLRVEDTAVYYCARGGSYWDYWGQGTLVTVSS

SEQ ID NO: 40 - DNA, 336 bp, Homo sapiens
gatattgtgatgacccagactccactcttctcatttgtcatgattggacagccggcctccatctcctgcaggtctaggcaaagcctcgtacacagtgatggaaacacctacttgaattggcttcagcagaggccaggccagcctccaagactcctaatttataagacttctaaccggttctctggggtcccagatagattcagtggcagtggggcagggacagatttcacactgaaaatcagcagggtggaagctgaggatgtcggggtttattactgtatgcaagctacacaatttcctatcacgttcggccaagggacacgactggagattaaa

SEQ ID NO: 41 - protein, 112 aa, Homo sapiens
DIVMTQTPLFSFVMIGQPASISCRSRQSLVHSDGNTYLNWLQQRPGQPPRLLIYKTSNRFSGVPDRFSGSGAGTDFTLKISRVEAEDVGVYYCMQATQFPITFGQGTRLEIK

SEQ ID NO: 42 - DNA, 370 bp, Homo sapiens
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagttgaggacacggctgtgtattactgtgcgaaagatcacggtgggaggtacgtctacgactacggtatggacgtctggggccaagggaccacggtcaccgtctcctcag

SEQ ID NO: 43 - protein, 123 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRVEDTAVYYCAKDHGGRYVYDYGMDVWGQGTTVTVSS

SEQ ID NO: 44 - DNA, 322 bp, Homo sapiens
gacatccagatgacccagtctccatcttccgtgtctgcatctgtaggagacagagtctccatcacttgtcgggcgagtcagggtattggcagctggttagcctggtatcagcagaaaccagggcaagcccctacgctcctaatctatgctgcctccagtttgcaacgtggggtcccatcaagattcagcggcagtggatctgggacagatttcactctcaccatcaacagcctgcagcctgaagattttgcaacttacttttgtcaacaggctaacagtttcccattcactttcggccctgggaccaaagtggatgtcaaac

SEQ ID NO: 45 - protein, 107 aa, Homo sapiens; identical to SEQ ID NO: 37

SEQ ID NO: 46 - protein, 34 aa, Homo sapiens
GASISSYYWSYIYYSGSTNYNPSLKSERLGIGDY

SEQ ID NO: 47 - protein, 41 aa, Homo sapiens
GFTFSSYGMHVISYDGSNKYYADSVKGDHGGRYVYDYGMDV

SEQ ID NO: 48 - protein, 41 aa, Homo sapiens; identical to SEQ ID NO: 47

SEQ ID NO: 49 - protein, 34 aa, Homo sapiens
GFTFSKYGMHVLWYDGSNKYYADSVKGDGHYFDY

SEQ ID NO: 50 - protein, 34 aa, Homo sapiens
GFTFSSYGMHVIWFDGNNKFYADSVKGAPAYWDY

SEQ ID NO: 51 - protein, 27 aa, Homo sapiens
RASQGIRNDLGAASSLQSLQHNTYPPT

SEQ ID NO: 52 - protein, 27 aa, Homo sapiens
RASQGISSWLAAASTLQRQQANSFPFT

SEQ ID NO: 53 - protein, 27 aa, Homo sapiens
RASQGISSWLVAASSLQRQQANSFPFT

SEQ ID NO: 54 - protein, 27 aa, Homo sapiens
RASQGISSWLAAASSLQRQQANSFPFT

SEQ ID NO: 55 - protein, 27 aa, Homo sapiens
RASQGIGSWLAAASSLQRQQANSFPFT

SEQ ID NO: 56 - protein, 32 aa, Homo sapiens
RSRQSLVHSDGNTYLNKTSNRFSMQATQFPIT

SEQ ID NO: 57 - protein, 28 aa, Homo sapiens
RASQSVSSSYLAGASSRATQQYGSSPWT

(19) United States
(12) Patent Application Publication
(10) Pub. No.: US 2004/0071694 A1
(43) Pub. Date: Apr. 15, 2004

(54) Erythropoietin receptor binding antibodies

(76) Inventors: Peter J. DeVries, Des Plaines, IL (US); Larry L. Green, San Francisco, CA (US); David H. Ostrow, Lake Zurich, IL (US); Edward B. Reilly, Libertyville, IL (US); James Wieler, Vancouver (CA)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/269,711

(22) Filed: Oct. 14, 2002

(51) Int. Cl. A61K 39/395; C12N 5/06; C07K 16/28

(52) U.S. Cl. 424/143.1; 530/388.22; 435/334

(57) ABSTRACT

The present invention relates to antibodies and antibody fragments thereof that bind to and activate an erythropoietin receptor. The present invention also relates to methods of modulating the endogenous activity of an erythropoietin receptor in a mammal using said antibodies as well as pharmaceutical compositions containing said antibodies.

### TECHNICAL FIELD OF THE INVENTION

The present invention relates to antibodies that recognize, bind to and, preferably, activate the erythropoietin receptor.

### BACKGROUND OF THE INVENTION

Erythropoietin (“EPO”) is a glycoprotein that is the primary regulator of erythropoiesis. Specifically, EPO is responsible for promoting the growth, differentiation and survival of erythroid progenitors, which give rise to mature red blood cells. In response to changes in the level of oxygen in the blood and tissues, erythropoietin appears to stimulate both proliferation and differentiation of immature erythroblasts. It also functions as a growth factor, stimulating the mitotic activity of erythroid progenitor cells, such as erythrocyte burst forming and colony-forming units. It also acts as a differentiation factor, triggering transformation of an erythrocyte colony-forming-unit into a proerythroblast (See Erslev, A., New Eng. J. Med., 316:101-103 (1987)).

EPO has a molecular weight of about 34,000 daltons and can occur in three forms—alpha, beta and asialo. During mid- to late gestation, EPO is synthesized in the fetal liver. Subsequently, EPO is synthesized in the kidney, circulates in the plasma and is excreted in the urine.

Human urinary EPO has been isolated and purified (See, Miyake et al., J. Biol. Chem., 252:5558 (1977)). Moreover, methods for identifying, cloning and expressing genes encoding EPO (See U.S. Pat. No. 4,703,008) as well as purifying recombinant EPO from a cell medium (See U.S. Pat. No. 4,667,016) are known in the art.

The activity of EPO is mediated through the binding and activation of a cell surface receptor referred to as the erythropoietin receptor. The EPO receptor belongs to the cytokine receptor superfamily and is believed to contain at least two distinct polypeptides, a 55-72 kDa species and a 85-100 kDa species (See U.S. Pat. No. 6,319,499, Mayeux et al., J. Biol. Chem, 266:23380 (1991), McCaffery et al., J. Biol. Chem., 264:10507 (1991)). Other studies have revealed other polypeptide complexes of EPO receptor having molecular weights such as 110, 130 and 145 kDa (See U.S. Pat. No. 6,319,499).

Both the murine and human EPO receptors have been cloned and expressed (See D'Andrea et al., Cell, 57:277 (1989); Jones et al., Blood, 76:31 (1990); Winkelmann et al., Blood, 76:24 (1990); WO 90/08822/U.S. Pat. No. 5,278,065). The full length human EPO receptor is a 483 amino acid transmembrane protein with an approximately 25 amino acid signal peptide (See U.S. Pat. No. 6,319,499). The human receptor demonstrates about a 82% amino acid sequence homology with the murine receptor. Id.

In the absence of ligand the EPO receptor exists in a preformed dimer. The binding of EPO to its receptor causes a conformational change such that the cytoplasmic domains are placed in close proximity. While not completely understood, it is believed that this “dimerization” plays a role in the activation of the receptor. The activation of the EPO receptor results in a number of biological effects. Some of these activities include stimulation of proliferation, stimulation of differentiation and inhibition of apoptosis (See U.S. Pat. No. 6,319,499, Liboi et al., PNAS USA, 90:11351 (1993), Koury, Science, 248:378 (1990)).

It is the relationship between the EPO receptor dimerization and activation that can be used to identify compounds (i.e. such as antibodies) other than EPO that are capable of: (1) dimerizing the EPO receptor; and (2) activating the receptor. These compounds would be useful in treating mammals suffering from anemia and in identifying mammals having a dysfunctional EPO receptor.

### SUMMARY OF THE INVENTION

In one embodiment, the invention relates to antibodies that bind to the human erythropoietin receptor. In one embodiment, the antibodies comprise a heavy chain variable region that is selected from the group consisting of SEQ ID NOS: 3, 7, 11, 15, 19, 23, 27, 31, 35, 39, 43 and fragments thereof. In another embodiment, the antibodies comprise a light chain variable region that is selected from the group consisting of SEQ ID NOS: 5, 9, 13, 17, 21, 25, 29, 33, 37, 41, 45 and fragments thereof.

In another embodiment, the present invention relates to an isolated antibody that is capable of binding a human erythropoietin receptor in a mammal. Such an antibody comprises a heavy chain variable region or a light chain variable region that comprises a continuous sequence from CDR1 through CDR3. The amino acid sequence of the heavy chain variable region comprising the continuous sequence from CDR1 through CDR3 is selected from the group consisting of: SEQ ID NO:46, SEQ ID NO:47, SEQ ID NO:48, SEQ ID NO:49, SEQ ID NO:50 and fragments thereof. The amino acid sequence of the light chain variable region comprising the continuous sequence from CDR1 through CDR3 is selected from the group consisting of: SEQ ID NO:51, SEQ ID NO:52, SEQ ID NO:53, SEQ ID NO:54, SEQ ID NO:55, SEQ ID NO:56, SEQ ID NO:57 and fragments thereof.

In another embodiment, the present invention relates to an antibody that activates an endogenous activity of a human erythropoietin receptor in a mammal but does not interact with a peptide having an amino acid sequence of PGNYSFSYQLEDEPWKLCRLHQAPTARGAV (SEQ ID NO:1).

In another embodiment, the present invention relates to an antibody that is capable of activating an endogenous activity of a human erythropoietin receptor in a mammal, wherein said antibody or antibody fragment thereof exhibits a binding affinity within one hundred fold of the binding affinity of endogenous human erythropoietin to the erythropoietin receptor.

In yet another embodiment, the present invention relates to an antibody or antibody fragment thereof that activates an endogenous activity of a human erythropoietin receptor in a mammal. The antibody or antibody fragment thereof comprises at least one human heavy chain variable region having the amino acid sequence of SEQ ID NO:3 or antibody fragment thereof, and/or at least one human light chain variable region having the amino acid sequence of SEQ ID NO:5 or antibody fragment thereof, provided that said antibody or antibody fragment thereof does not interact with a peptide having an amino acid sequence of PGNYSFSYQLEDEPWKLCRLHQAPTARGAV (SEQ ID NO:1).

In yet another embodiment, the present invention relates to an antibody or antibody fragment thereof that activates an endogenous activity of a human erythropoietin receptor in a mammal. The antibody or antibody fragment thereof comprises at least one heavy chain variable region having the amino acid sequence of SEQ ID NO:7 or antibody fragment thereof, and/or at least one light chain variable region having the amino acid sequence of SEQ ID NO:9 or antibody fragment thereof, provided that said antibody or antibody fragment thereof does not interact with a peptide having an amino acid sequence of PGNYSFSYQLEDEPWKLCRLHQAPTARGAV (SEQ ID NO:1).

In yet another embodiment, the present invention relates to a method of activating an endogenous activity of a human erythropoietin receptor in a mammal. The method involves the step of administering to a mammal a therapeutically effective amount of an antibody or antibody fragment thereof to activate the EPO receptor. The antibody or antibody fragment thereof does not interact with a peptide having an amino acid sequence of PGNYSFSYQLEDEPWKLCRLHQAPTARGAV (SEQ ID NO:1).

In yet a further embodiment, the present invention relates to a method of modulating an endogenous activity of a human erythropoietin receptor in a mammal. The method involves administering to a mammal a therapeutically effective amount of an antibody or antibody fragment thereof to modulate the endogenous activity of a human erythropoietin receptor in a mammal but does not interact with a peptide having an amino acid sequence of PGNYSFSYQLEDEPWKLCRLHQAPTARGAV (SEQ ID NO:1).

In yet a further embodiment, the present invention relates to a method of treating a mammal suffering from pure red cell aplasia induced by neutralizing anti-erythropoietin antibodies. The method involves administering to a mammal in need of treatment a therapeutically effective amount of an antibody or antibody fragment thereof to activate said receptor, wherein said antibody or antibody fragment thereof does not interact with a peptide having an amino acid sequence of PGNYSFSYQLEDEPWKLCRLHQAPTARGAV (SEQ ID NO:1).

In yet a further embodiment, the present invention relates to pharmaceutical compositions. The pharmaceutical compositions of the present invention contain a therapeutically effective amount of a pharmaceutically acceptable excipient and an antibody or antibody fragment thereof. The antibody or antibody fragment contained in the pharmaceutical composition activates an endogenous activity of a human erythropoictin receptor in a mammal but does not interact with a peptide having an amino acid sequence of PGNYSFSYQLEDEPWKLCRLHQAPTARGAV (SEQ ID NO:1).

Finally, the present invention relates to isolated and purified polynucleotide and amino acid sequences. The isolated and purified polynucleotide sequences can be selected from the group consisting of: SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO: 18, SEQ ID NO:20, SEQ ID NO:22, SEQ ID NO:24, SEQ ID NO:26, SEQ ID NO:28,SEQ ID NO:30, SEQ ID NO:32, SEQ ID NO:34, SEQ ID NO:36, SEQ ID NO:38, SEQ ID NO:40, SEQ ID NO:42 AND SEQ ID NO:44 and fragments, complements and degenerate codon equivalents thereof. The present invention further relates to isolated and purified amino acid sequences selected from the group consisting of: SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:46, SEQ ID NO:47, SEQ ID NO:48, SEQ ID NO:49, SEQ ID NO:50, SEQ ID NO:51, SEQ IDNO:52, SEQ ID NO:53, SEQ ID NO:54, SEQ ID NO:55, SEQ ID NO:56, SEQ ID NO:57 and fragments and complements and thereof.

### BRIEF DESCRIPTION OF THE FIGURES

FIG. 1 shows the isolated and purified polynucleotide and amino acid sequence of the heavy chain of human antibody Ab12. The variable chain ends at nucleotide 1385. The variable/constant joining region (underlined) is at nucleotides 1386-1391. The constant region is from nucleotides 1392-2928.

FIG. 2 shows the isolated and purified polynucleotide and amino acid sequence of the light chain of human antibody Ab12. The variable chain ends at nucleotide 1363. The variable/constant joining region (underlined) is at nucleotides 1364-1369. The constant region is from nucleotides 1370-1618.

FIG. 3 shows the isolated and purified polynucleotide and amino acid sequence of the heavy chain of human antibody Ab198. The variable chain ends at nucleotide 1304. The variable/constant joining region (underlined) is at nucleotides 1305-1310. The constant region is from nucleotides 1311-2847.

FIG. 4 shows the isolated and purified polynucleotide and amino acid sequence of the light chain of human antibody Ab198. The variable chain ends at nucleotide 1363. The variable/constant joining region (underlined) is at nucleotides 1364-1370. The constant region is from nucleotides 1371-1618.

FIG. 5 shows the competition of Ab12 with ¹²⁵I-labeled EPO for binding to Chinese Hamster Ovary cells expressing recombinant EPO receptor.

FIG. 6 shows the results of an EPO dependent human cell proliferation assay using Ab12 and Ab198.

FIG. 7 shows that Ab12 remains active in inducing the proliferation of F36E cells after storage at 4° C. for up to 20 days.

FIG. 8 shows that Ab12 induces the formation of CFU-E (colony forming unit-erythroid) from human 36⁺ progenitor cells.

FIG. 9 shows the induction of proliferation of human erythroid producing cells with Ab198.

FIG. 10 shows that Ab198 induces the formation of CFU-E colonies from cynomologous bone marrow-derived erythroid progenitor cells.

FIG. 11 shows that Ab12 does not interact with the peptide SE-3. Ab71A interacts with the SE-3 peptide.

FIG. 12 shows that human Abs secreted by primary hybridomas induce the proliferation of F36E cells.

FIG. 13 shows that human Ab supernatants secreted by primary hybridomas interact with intact EPO receptor, but not with peptide SE-3.

FIG. 14 shows the activity of various concentrations of Ab12 on the proliferation of UT7/EPO cells.

FIG. 15 shows the activity of various concentrations of Ab198 on the proliferation of UT7/EPO cells.

FIG. 16 shows the activity of various concentrations of Ab198 (with or without the addition of a secondary goat anti-human FC antibody) on the growth and proliferation of UT7/EPO cells.

FIG. 17 shows the activity of various concentrations of Ab12 (with or without the addition of a secondary goat anti-human FC antibody) on the growth and proliferation of UT7/EPO cells.

FIG. 18 is a series of representations of the heavy chain and light chain variable region nucleotide and amino acid sequences of the human anti-EPO-R antibody expressed by the cell line designated ABT2-SCX-003 of the invention, with FIG. 18A representing the nucleotide sequence encoding the variable region of the heavy chain, FIG. 18B representing the amino acid sequence encoded by the nucleotide sequence shown in FIG. 18A, FIG. 18C representing the nucleotide sequence encoding the variable region of the light chain, and FIG. 18D representing the amino acid sequence encoded by the nucleotide sequence shown in FIG. 18C.

FIG. 19 is a series of representations of the heavy chain and light chain variable region nucleotide and amino acid sequences of the human anti-EPO-R antibody expressed by the cell line designated ABT2-SCX-012 (also referred to herein as Ab12) of the invention, with FIG. 19A representing the nucleotide sequence encoding the variable region of the heavy chain, FIG. 19B representing the amino acid sequence encoded by the nucleotide sequence shown in FIG. 19A, FIG. 19C representing the nucleotide sequence encoding the variable region of the light chain, and FIG. 19D representing the amino acid sequence encoded by the nucleotide sequence shown in FIG. 19C.

FIG. 20 is a series of representations of the heavy chain and light chain variable region nucleotide and amino acid sequences of the human anti-EPO-R antibody expressed by the cell line designated ABT2-SCX-022 of the invention, with FIG. 20A representing the nucleotide sequence encoding the variable region of the heavy chain, FIG. 20B representing the amino acid sequence encoded by the nucleotide sequence shown in FIG. 20A, FIG. 20C representing the nucleotide sequence encoding the variable region of the light chain, and FIG. 20D representing the amino acid sequence encoded by the nucleotide sequence shown in FIG. 20C.

FIG. 21 is a series of representations of the heavy chain and light chain variable region nucleotide and amino acid sequences of the human anti-EPO-R antibody expressed by the cell line designated ABT2-SCX-054 of the invention, with FIG. 21A representing the nucleotide sequence encoding the variable region of the heavy chain, FIG. 21B representing the amino acid sequence encoded by the nucleotide sequence shown in FIG. 21A, FIG. 21C representing the nucleotide sequence encoding the variable region of the light chain, and FIG. 21D representing the amino acid sequence encoded by the nucleotide sequence shown in FIG. 21C.

FIG. 22 is a series of representations of the heavy chain and light chain variable region nucleotide and amino acid sequences of the human anti-EPO-R antibody expressed by the cell line designated ABT2-SCX-060 of the invention, with FIG. 22A representing the nucleotide sequence encoding the variable region of the heavy chain, FIG. 22B representing the amino acid sequence encoded by the nucleotide sequence shown in FIG. 22A, FIG. 22C representing the nucleotide sequence encoding the variable region of the light chain, and FIG. 22D representing the amino acid sequence encoded by the nucleotide sequence shown in FIG. 22C.

FIG. 23 is a series of representations of the heavy chain and light chain variable region nucleotide and amino acid sequences of the human anti-EPO-R antibody expressed by the cell line designated ABT2-SCX-102 of the invention, with FIG. 23A representing the nucleotide sequence encoding the variable region of the heavy chain, FIG. 23B representing the amino acid sequence encoded by the nucleotide sequence shown in FIG. 23A, FIG. 23C representing the nucleotide sequence encoding the variable region of the light chain, and FIG. 23D representing the amino acid sequence encoded by the nucleotide sequence shown in FIG. 23C.

FIG. 24 is a series of representations of the heavy chain and light chain variable region nucleotide and amino acid sequences of the human anti-EPO-R antibody expressed by the cell line designated ABT2-SCX-135 of the invention, with FIG. 24A representing the nucleotide sequence encoding the variable region of the heavy chain, FIG. 24B representing the amino acid sequence encoded by the nucleotide sequence shown in FIG. 24A, FIG. 24C representing the nucleotide sequence encoding the variable region of the light chain, and FIG. 24D representing the amino acid sequence encoded by the nucleotide sequence shown in FIG. 24C.

FIG. 25 is a series of representations of the heavy chain and light chain variable region nucleotide and amino acid sequences of the human anti-EPO-R antibody expressed by the cell line designated ABT2-SCX-145 of the invention, with FIG. 25A representing the nucleotide sequence encoding the variable region of the heavy chain, FIG. 25B representing the amino acid sequence encoded by the nucleotide sequence shown in FIG. 25A, FIG. 25C representing the nucleotide sequence encoding the variable region of the light chain, and FIG. 25D representing the amino acid sequence encoded by the nucleotide sequence shown in FIG. 25C.

FIG. 26 is a series of representations of the heavy chain and light chain variable region nucleotide and amino acid sequences of the human anti-EPO-R antibody expressed by the cell line designated ABT2-SCX-198 (also referred to herein as Ab198) of the invention, with FIG. 26 representing the nucleotide sequence encoding the variable region of the heavy chain, FIG. 26B representing the amino acid sequence encoded by the nucleotide sequence shown in FIG. 26A, FIG. 26C representing the nucleotide sequence encoding the variable region of the light chain, and FIG. 26D representing the amino acid sequence encoded by the nucleotide sequence shown in FIG. 26C.

FIG. 27 is a series of representations of the heavy chain and light chain variable region nucleotide and amino acid sequences of the human anti-EPO-R antibody expressed by the cell line designated ABT2-SCX-254 of the invention, with FIG. 27A representing the nucleotide sequence encoding the variable region of the heavy chain, FIG. 27B representing the amino acid sequence encoded by the nucleotide sequence shown in FIG. 27A, FIG. 27C representing the nucleotide sequence encoding the variable region of the light chain, and FIG. 27D representing the amino acid sequence encoded by the nucleotide sequence shown in FIG. 27C.

FIG. 28 is a series of representations of the heavy chain and light chain variable region nucleotide and amino acid sequences of the human anti-EPO-R antibody expressed by the cell line designated ABT2-SCX-267 of the invention, with FIG. 28A representing the nucleotide sequence encoding the variable region of the heavy chain, FIG. 28B representing the amino acid sequence encoded by the nucleotide sequence shown in FIG. 28A, FIG. 28C representing the nucleotide sequence encoding the variable region of the light chain, and FIG. 28D representing the amino acid sequence encoded by the nucleotide sequence shown in FIG. 28C.

FIG. 29 is a table showing amino acid sequence alignments of heavy chain variable regions of anti-EPOr mAbs generated according to the invention with their associated germline variable region sequences and identifying framework regions and complementarity determining regions.

FIG. 30 is a table showing amino acid sequence alignments of light chain variable regions of anti-EPOr mAbs generated according to the invention with their associated germline variable region sequences and identifying framework regions and complementarity determining regions.

### DETAILED DESCRIPTION OF THE INVENTION

Definitions

As used herein, the term “antibody” or “immunoglobulin” refers to single chain, two-chain, and multi-chain proteins and glycoproteins that belong to the classes of polyclonal, monoclonal, chimeric and human or humanized. The term “antibody” also includes synthetic and genetically engineered variants thereof.

As used herein, the term “antibody fragment” refers to Fab, Fab′, F(ab′)₂ and Fv fragments, as well as any portion of an antibody having specificity toward at least one desired epitope.

As used herein, the term “humanized antibody” refers to an antibody that is derived from a non-human antibody (i.e murine) that retains or substantially retains the antigen-binding properties of the parent antibody but is less immunogenic in humans.

As used herein, the term “human antibody” refers to an antibody that possesses a sequence that is derived from a human germ-line immunoglobulin sequence, such as antibodies derived from transgenic mice having human immunoglobulin genes (e.g., XenoMouse® mice), human phage display libraries, or human B cells.

As used herein, the term “epitope” refers to any protein determinate capable of specifically binding to an antibody or T-cell receptors. Epitopic determinants usually consist of chemically active surface groupings of molecules such as amino acids or sugar side chains and usually have specific three dimensional structural characteristics, as well as specific charge characteristics.

As used herein, the term “endogenous” refers to a product or activity arising in the body or cell as opposed to a product or activity coming from outside.

As used herein the phrase, a polynucleotide “derived from” or “specific for a designated sequence refers to a polynucleotide sequence that comprises a contiguous sequence of approximately at least 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The sequence may be complementary or identical to a sequence that is unique to a particular polynucleotide sequence as determined by techniques known in the art. Regions from which sequences may be derived, include but are not limited to, regions encoding specific epitopes, as well as non-translated and/or non-transcribed regions.

The derived polynucleotide will not necessarily be derived physically from the nucleotide sequence of interest under study, but may be generated in any manner, including, but not limited to, chemical synthesis, replication, reverse transcription or transcription, that is based on the information provided by the sequence of bases in the region(s) from which the polynucleotide is derived. As such, it may represent either a sense or an antisense orientation of the original polynucleotide. In addition, combinations of regions corresponding to that of the designated sequence may be modified in ways known in the art to be consistent with the intended use.

As used herein, the phrase “encoded by” refers to a nucleic acid sequence that codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. Also encompassed are polypeptide sequences that are immunologically identifiable with a polypeptide encoded by the sequence. Thus, a “polypeptide,” “protein” or “amino acid” sequence has at least about 50%, 60%, 70%, 75%, 80%, 85%, 90%, 95% or more identity to the antibodies of the present invention. Further, the antibodies of the present invention may have at least about 60%, 70%, 75%, 80%, 85%, 90% or 95% similarity to a polypeptide or amino sequences of the antibodies of the present invention. The amino acid sequences of the antibodies of the present invention can be selected from the group consisting of SEQUENCE ID NOS: 3, 5, 7 and 9.

As used herein, the phrase “recombinant polypeptide,” “recombinant protein,” or “a polypeptide produced by recombinant techniques”, which terms may be used interchangeably herein, describes a polypeptide that by virtue of its origin or manipulation is not associated with all or a portion of the polypeptide with which it is associated in nature and/or is linked to a polypeptide other than that to which it is lined in nature. A recombinant or encoded polypeptide or protein is not necessarily translated from a designated nucleic acid sequence. It also may be generated in any manner, including chemical synthesis or expression of a recombinant expression system.

As used herein, the phrase “synthetic peptide” refers to a polymeric form of amino acids of any length, which may be chemically synthesized by methods well-known in the art (See U.S. Pat. Nos. 4,816,513, 5,854,389, 5,891,993 and 6,184,344).

As used herein, the term “polynucleotide” refers to a polymeric form of nucleotides of any length, either ribonucleotides or deoxyribonucleotides. This term refers only to the primary structure of the molecule. Thus, the term includes double and single-stranded DNA as well as double- and single-stranded RNA. It also includes modifications, such as methylation or capping and unmodified forms of the polynucleotide. The terms “polynucleotide”, “oligomer,” “oligonucleotide,” and “oligo,” are used interchangeably herein.

As used herein the phrase “purified polynucleotide” refers to a polynucleotide of interest or fragment thereof that is essentially free, e.g. contains less than about 50%, 60%, 70%, 75%, 80%, 85%, 90%, 95% of the protein with which the polynucleotide is naturally associated. Techniques for purifying polynucleotides of interest are well known in the art and include, for example, disruption of the cell containing the polynucleotide with a chaotropic agent and separation of the polynucleotide(s) and proteins by ion-exchange chromatography, affinity chromatography and sedimentation according to density.

As used herein, the phrase “purified polypeptide” or “purified protein” means a polypeptide of interest or fragment thereof which is essentially free of, e.g., contains less than about 50%, 60%, 70%, 75%, 80%, 85%, 90%, 95%, cellular components with which the polypeptide of interest is naturally associated. Methods for purifying polypeptides of interest are known in the art.

As used herein, the term “isolated” refers to material that is removed from its original environment (e.g., the natural environment if it is naturally occurring). For example, a naturally-occurring polynucleotide or polypeptide present in a living animal is not isolated, but the same polynucleotide or DNA or polypeptide, that is separated from some or all of the coexisting materials in the natural system, is isolated. Such polynucleotide could be part of a vector and/or such polynucleotide or polypeptide could be part of a composition, and still be isolated in that the vector or composition is not part of its natural environment.

As used herein, the term “polypeptide” and “protein” are used interchangeably and refer to at least one molecular chain of amino acids linked through covalent and/or non-covalent bonds. The terms do not refer to a specific length of the product. Thus, peptides, oligopeptides and proteins are included within the definition of polypeptide. The terms include post-translational modifications of the polypeptide, including, but not limited to, glycosylations, acetylations, phosphorylations and the like. In addition, protein fragments, analogs, mutated or variant proteins, fusion proteins and the like are included within the meaning of polypeptide.

As used herein, the phrase “recombinant host cells,” “host cells,” “cells,” “cell lines,” “cell cultures,” and other such terms denoting microorganisms or higher eukaryotic cell lines cultured as unicellular entities refer to cells that can be, or have been, used as recipients for recombinant vector or other transferred DNA, and include the original progeny of the original cell that has been transfected.

As used herein, the term “replicon” refers to any genetic element, such as a plasmid, a chromosome or a virus, that behaves as an autonomous unit of polynucleotide replication within a cell.

As used herein, the term “operably linked” refers to a situation wherein the components described are in a relationship permitting them to function in their intended manner. Thus, for example, a control sequence “operably linked” to a coding sequence is ligated in such a manner that expression of the coding sequence is achieved under conditions compatible with the control sequence.

As used herein, the term “vector” refers to a replicon in which another polynucleotide segment is attached, such as to bring about the replication and/or expression of the attached segment.

As used herein, the term “control sequence” refers to a polynucleotide sequence that is necessary to effect the expression of a coding sequence to which it is ligated. The nature of such control sequences differs depending upon the host organism. In prokaryotes, such control sequences generally include a promoter, a ribosomal binding site and terminators and, in some instances, enhancers. The term “control sequence” thus is intended to include at a minimum all components whose presence is necessary for expression, and also may include additional components whose presence is advantageous, for example, leader sequences.

The term “transfection” refers to the introduction of an exogenous polynucleotide into a prokaryotic or eucaryotic host cell, irrespective of the method used for the introduction. The term “transfection” refers to both stable and transient introduction of the polynucleotide, and encompasses direct uptake of polynucleotides, transformation, transduction and f-mating. Once introduced into the host cell, the exogenous polynucleotide may be maintained as a non-integrated replicon, for example, a plasmid, or alternatively, may be integrated into the host genome.

As used herein, the term “treatment” refers to prophylaxis and/or therapy.

As used herein, the term “purified product” refers to a preparation of the product which has been isolated from the cellular constituents with which the product is normally associated and from other types of cells that may be present in the sample of interest.

As used herein, the phrase “activation of an erythropoietin (EPO) receptor” refers to one or more molecular processes which an EPO receptor undergoes that result in the transduction of a signal to the interior of a receptor-bearing cell. Ultimately, this signal brings about one or more changes in cellular physiology. Activation of the EPO receptor typically results in the proliferation or differentiation of EPO receptor-bearing cells, such as, but not limited to, erythroid progenitor cells. A number of events are involved in the activation of the EPO receptor, such as, but not limited to, the dimerization of the receptor.

Introduction

The structural unit of an antibody is a tetramer. Each tetramer is composed of two identical pairs of polypeptide chains, each pair having one “light” (25 kDa) and one “heavy” chain (about 50-70 kDa). The amino-terminal portion of each chain includes a variable region that is primarily responsible for antigen recognition. The carboxy-terminal portion of the chain defines a constant region that is responsible for the effector function of the antibody. Human light chains are classified as kappa and lambda light chains. Heavy chains are classified as mu, delta, gamma, alpha, or epsilon and define the antibody's isotype as IgM, IgD, IgG, IgA, and IgE. Within the light and heavy chains, the variable and constant regions are joined by a “J” region with the heavy chain also include a “D” region. The variable regions of each light/heavy chain pair form the antigen binding site. Thereupon, an intact antibody has two binding sites, which, except in bifunctional or bispecific antibodies, are the same. Bifunctional or bispecific antibodies are artificial hybrid antibodies that have two different heavy/light chain pairs and two different binding sites. Bifunctional or bispecific antibodies can be produced using routine techniques known in the art.

The structure of the chains of an antibody exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both the light and heavy chains comprise the domains FR1, CDR1, FR2, CDR2, FR3, CDR3 and FR4.

U.S. Pat. No. 6,319,499 describes antibodies that bind to and activate an erythropoietin receptor (EPO-R). The antibodies specifically identified in this patent are Mabs 71 and 73. Mab 71 binds to a peptide designated “SE-3” having the amino acid sequence of PGNYSFSYQLEDEPWKLCRLHQAPTARGAV (SEQ ID NO:1) (See Example 3). SE-3 is located on the human EPO-R between amino acid residues 49-78. According to U.S. Pat. No. 6,319,499, when this region of the EPO-R (i.e. amino acid residues 49-79) is bound with a cross linker such as Mab 71, this results in the activation of the EPO receptor. Example 6 in U.S. Pat. No. 6,319,499 states that Mab 71 binds “significant amounts of peptide SE-3” compared to other peptides tested. This example further states that this “indicates that Mab 71 binds to a region of the human EPO-R containing or overlapping residues 49 to 78.”

In one embodiment, the present invention relates to an antibody or antibody fragment that binds to the erythropoietin receptor. The antibody or antibody fragment that binds to the erythropoietin receptor comprises at least one heavy chain having an amino acid sequence selected from the group consisting of: SEQ ID NOS: 3, 7, 11, 15, 19, 23, 27, 31, 35, 39, 43 and fragments thereof. In a second embodiment, the antibody or antibody fragment that binds to the erythropoietin receptor comprises at least one light chain having an amino acid sequence selected from the group consisting of: SEQ ID NOS: 5, 9, 13, 17, 21, 25, 29, 33, 37, 41, 45 and fragments thereof.

In a third embodiment, the present invention relates to an isolated antibody that is capable of binding a human erythropoietin receptor in a mammal. More specifically, the antibody comprises a heavy chain variable region or a light chain variable region which comprises a continuous sequence from CDR1 through CDR3. The amino acid sequence of the heavy chain variable region comprising the continuous sequence from CDR1 through CDR3 is selected from the group consisting of: SEQ ID NO:46, SEQ ID NO:47, SEQ ID NO:48, SEQ ID NO:49, SEQ ID NO:50 and fragments thereof. The amino acid sequence of the light chain variable region comprising the continuous sequence from CDR1 through CDR3is selected from the group consisting of: SEQ ID NO:51, SEQ ID NO:52, SEQ ID NO:53, SEQ ID NO:54, SEQ ID NO:55, SEQ ID NO:56, SEQ ID NO:57 and fragments thereof.

In a fourth embodiment, the present invention relates to an antibody or antibody fragment that binds to and activates the erythropoietin receptor. The antibodies of the present invention bind to at least one epitope that is involved in activating the EPO receptor (Example 4). Unlike other antibodies or fragments known in the art that bind to and activate an erythropoietin receptor, such as the antibodies described in U.S. Pat. No. 6,319,499, the antibodies of the present invention do not interact with the peptide designated SE-3. Surprisingly, the antibodies of the present invention are erythropoietic even though the antibodies do not bind to the SE-3 peptide. Therefore, the human antibodies of the present invention interact with at least one different epitope on the human EPO receptor than the antibodies described in U.S. Pat. No. 6,319,499.

Additionally, as demonstrated by the BlAcore results shown in Example 1, the antibodies of the present invention exhibit a binding affinity to the erythropoietin receptor within one hundred fold of the binding affinity of endogenous human erythropoietin to the erythropoietin receptor. A high (˜1 nM) and low (˜1 μM) affinity of the EPO receptor for EPO has been reported resulting from two non equivalent receptor binding sites on EPO (See Philo, J. S. et al., Biochemistry, 35:1681 (1996)).

The antibodies of the present invention can be polyclonal antibodies, monoclonal antibodies, chimeric antibodies (See U.S. Pat. No. 6,020,153) or human or humanized antibodies or antibody fragments thereof. Synthetic and genetically engineered variants (See U.S. Pat. No. 6,331,415) of any of the foregoing are also contemplated by the present invention. Preferably, however, the antibodies of the present invention are human or humanized antibodies. The advantage of human or humanized antibodies is that they potentially decrease or eliminate the immunogenicity of the antibody in a host recipient, thereby permitting an increase in the bioavailability and a reduction in the possibility of adverse immune reaction, thus potentially enabling multiple antibody administrations.

Humanized antibodies include chimeric or CDR-grafted antibodies. Also, human antibodies can be produced using genetically engineered strains of animals in which the antibody gene expression of the animal is suppressed and functionally replaced with human antibody gene expression.

Methods for making humanized and human antibodies are known in the art. One method for making human antibodies employs the use of transgenic animals, such as a transgenic mouse. These transgenic animals contain a substantial portion of the human antibody producing genome inserted into their own genome and the animal's own endogenous antibody production is rendered deficient in the production of antibodies. Methods for making such transgenic animals are known in the art. Such transgenic animals can be made using XenoMouse® technology or by using a “minilocus” approach. Methods for making Xenomice™ are described in U.S. Pat. Nos. 6,162,963, 6,150,584, 6,114,598 and 6,075,181. Methods for making transgenic animals using the “minilocus” approach are described in U.S. Pat. Nos. 5,545,807, 5,545,806 and 5,625,825.

Using the XenoMouse® technology, human antibodies can obtained by immunizing a XenoMouse® mouse with an antigen of interest. The lymphatic cells (such as B-cells) are recovered from the mice that express antibodies. These recovered cells can be fused with myeloid-type cell line to prepare immortal hybridoma cell lines. These hybridoma cell lines can be screened and selected to identify hybridoma cell lines that produce antibodies specific to the antigen of interest. Alternatively, the antibodies can be expressed in cell lines other than hybridoma cell lines. More specifically, sequences encoding particular antibodies can be cloned from cells producing the antibodies and used for transformation of a suitable mammalian host cell. Transformation can be by any known method for introducing polynucleotides into a host cell, including, for example, packaging the polynucleotide in a virus or into a viral vector and transducing a host cell with a virus or vector or by transfection procedures known in the art such as those described in U.S. Pat. Nos. 4,399,216, 4,912,040, 4,740,461 and 4,959,455. For example, one or more genes encoding the heavy chain can be expressed in a cell and one or more genes encoding the light chain can be expressed in a second cell. The resulting heavy and light chains can then be fused together to form the antibodies of the present invention using techniques known in the art. Alternatively, genes encoding for parts of the heavy and light chains can be ligated using restriction endonucleases to reconstruct the gene coding for each chain. Such a gene can then be expressed in a cell to produce the antibodies of the present invention.

The transformation procedure used will depend upon the host to be transformed. Methods for introducing heterologous polynucleotides into mammalian cells are well known in the art and include dextran-mediated transfection, calcium phosphate precipitation, polybrene mediated transfection, protoplast fusion, electroporation, encapsulation of the polynucleotide(s) into liposomes and direct microinjection of the DNA molecule.

Mammalian cell lines that can be used as hosts for expression are well known in the art and include, but are not limited to, Chinese hamster ovary (CHO) cells, HeLa cells, baby hamster kidney (BHK) cells, monkey kidney cells (COS), human hepatocellular carcinoma cells bacterial cells, such as E. coli, yeast cells, such as Saccharomyces cerevisiae, etc.

Humanized antibodies can also be made using a CDR-grafted approach. Such humanized antibodies are well known in the art. Generally, humanized antibodies are produced by obtaining nucleic acid sequences that encode the variable heavy and variable light sequences of an antibody that binds to the EPO receptor, identifying the complementary determining region or “CDR” in the variable heavy and variable light sequences and grafting the CDR nucleic acid sequences on to human framework nucleic acid sequences.

The human framework that is selected is one that is suitable for in vivo administration, meaning that it does not exhibit immunogenicity. For example, such a determination can be made by prior experience with in vivo usage of such antibodies and studies of amino acid similarities.

Methods for cloning nucleic acids are known in the art. These methods involve amplification of the antibody sequences to be cloned using appropriate primers by polymerase chain reaction (PCR). Primers that are suitable for amplifying antibody nucleic acid sequences and specifically murine variable heavy and variable light sequences are known in the art.

Once the CDRs and FRs of the cloned antibody sequences that are to be humanized are identified, the amino acid sequences encoding the CDRs are identified and the corresponding nucleic acid sequences grafted on to selected human FRs. This can be done using known primers and linkers, the selection of which are known in the art.

After the CDRs are grafted onto selected human FRs, the resulting “humanized” variable heavy and variable light sequences are expressed to produce a humanized Fv or humanized antibody that binds to the EPO receptor. Typically, the humanized variable heavy and light sequences are expressed as a fusion protein with human constant domain sequences so an intact antibody that binds to the EPO receptor is obtained. However, a humanized Fv antibody can be produced that does not contain the constant sequences. Fusion of the human constant sequence to the humanized variable region is preferred.

The EPO receptor that is bound by and preferably activated using the antibodies of the present invention is preferably a mammalian EPO receptor, most preferably a human EPO receptor. The present invention also contemplates the use of analogs of the EPO receptor, such as those described in U.S. Pat. No. 5,292,654. Human EPO receptor can be purchased from R & D Systems (Minneapolis, Minn.).

An example of two (2) antibodies that (1) bind to and activate the EPO receptor; (2) do not interact with a peptide having an amino acid sequence of PGNYSFSYQLEDEPWKLCRLHQAPTARGAV (SEQ ID NO: 1); and (3) exhibit a binding affinity within one hundred fold of the binding affinity of endogeous human EPO to the EPO receptor, are the human antibodies designated Ab12 and Ab198. Ab12 and Ab198 are human antibodies that were developed using the XenoMouse® XenoMax technology described herein (See Example 1).

In another embodiment, the present invention relates to polynucleotide and polypeptide sequences that encode for the antibodies described herein. Preferably, such polynucleotides encode for both the variable and constant regions of each of the heavy and light chains, although other combinations are also contemplated by the present invention.

The present invention also contemplates oligonucleotide fragments derived from the disclosed polynucleotides and nucleic acid sequences complementary to these polynucleotides. The polynucleotides can be in the form of RNA or DNA. Polynucleotides in the form of DNA, cDNA, genomic DNA, nucleic acid analogs and synthetic DNA are within the scope of the present invention. The DNA may be double-stranded or single-stranded, and if single stranded, may be the coding (sense) strand or non-coding (anti-sense) strand. The coding sequence that encodes the polypeptide may be identical to the coding sequence provided herein or may be a different coding sequence which coding sequence, as a result of the redundancy or degeneracy of the genetic code, encodes the same polypeptide as the DNA provided herein.

Preferably, the polynucleotides encode at least one heavy chain variable region and at least one light chain variable region of the present invention. Examples of such polynucleotides are shown in SEQ ID NOS: 2, 4, 6, 8, 10, 12, 14, 16, 18, 20, 22, 24, 26, 28, 30, 32, 34, 36, 38, 40, 42, 44 as well as fragments, complements and degenerate codon equivalents thereof. For example, SEQ ID NO: 2 encodes for the heavy chain of Ab12 (variable region) and SEQ ID NO:4 encodes for the light chain of Ab12 (variable region). SEQ ID NO:6 encodes for the heavy chain of Ab198 (variable region) and SEQ ID NO: 8 encodes for the light chain of Ab198 (variable region).

The present invention also includes variant polynucleotides containing modifications such as polynucleotide deletions, substitutions or additions, and any polypeptide modification resulting from the variant polynucleotide sequence. A polynucleotide of the present invention may also have a coding sequence that is a naturally occurring variant of the coding sequence provided herein.

It is contemplated that polynucleotides will be considered to hybridize to the sequences provided herein if there is at least 50%, 60%, 70%, 75%, 80%, 85%, 90%, 95% identity between the polynucleotide and the sequence.

The present invention further relates to polypeptides that encode for the antibodies of the present invention as well as fragments, analogs and derivatives of such polypeptides. The polypeptides of the present invention may be recombinant polypeptides, naturally purified polypeptides or synthetic polypeptides. The fragment, derivative or analogs of the polypeptides of the present invention may be one in which one or more of the amino acid residues is substituted with a conserved or non-conserved amino acid residue (preferably a conserved amino acid residue) and such substituted amino acid residue may or may not be one encoded by the genetic code; or it may be one in which one or more of the amino acid residues includes a substitutent group; or it may be on in which the polypeptide is fused with another compound, such as a compound to increase the half-life of the polypeptide (for example, polyethylene glycol); or it may be one in which the additional amino acids are fused to the polypeptide, such as a leader or secretory sequence or a sequence that is employed for purification of the polypeptide or a proprotein sequence. Such fragments, derivatives and analogs are within the scope of the present invention.

A polypeptide of the present invention may have an amino acid sequence that is identical to that of the antibodies described herein or that is different by minor variations due to one or more amino acid substitutions. The variation may be a “conservative change” typically in the range of about 1 to 5 amino acids, wherein the substituted amino acid has similar structural or chemical properties, e.g., replacement of leucine with isoleucine or threonine with serine. In contrast, variations may include nonconservative changes, e.g., replacement of a glycine with a tryptophan. Similar minor variations may also include amino acid deletions or insertions or both. Guidance in determining which and how many amino acid residues may be substituted, inserted, or deleted without changing biological or immunological activity may be found using computer programs well known in the art, for example DNASTAR software (DNASTAR, Inc., Madison, Wis.).

Preferably, the polypeptides encode at least one heavy chain variable region, at least one light chain variable region of the antibodies of the present invention. Examples of such polypeptides are those having the amino acid sequences shown in SEQ ID NOS: 3, 5, 7, 9, 11, 13, 15, 17, 19, 21, 23, 25, 27, 29, 31, 33, 35, 37, 39, 41, 43, 45, 46, 47, 48, 49, 50, 51, 52, 53, 54, 55, 56, 57 and fragments thereof. Specifically, the heavy chain of Ab12 has the amino acid sequence shown in SEQ ID NO: 3 and the light chain has the amino acid sequence shown in SEQ ID NO:5. The amino acid sequence of the heavy chain of Ab198 is shown in SEQ ID NO:7 and the light chain has the amino acid sequence shown in SEQ ID NO:9.

The present invention also provides vectors that include the polynucleotides of the present invention, host cells which are genetically engineered with vectors of the present invention and the production of the antibodies of the present invention by recombinant techniques.

Host cells are genetically engineered (transfected, transduced or transformed) with vectors, such as, cloning vectors or expression vectors. The vector may be in the form of a plasmid, a viral particle, a phage, etc. The engineered host cells can be cultured in conventional nutrient media modified as appropriate for activating promoters, selecting transfected cells, etc. The culture conditions, such as temperature, pH and the like, are those previously used with the host cell selected for expression, and will be apparent to those of skilled in the art.

The polynucleotides of the present invention can be employed to produce the polypeptides and hence the antibodies of the present invention. The polynucleotide sequences of the present invention can be included in any one of a variety of expression vehicles, in particular, vectors or plasmids for expressing a polypeptide. Such vectors include chromosomal, nonchromosomal and synthetic DNA sequences, derivatives of SV40, bacterial plasmids, phage DNA, yeast plasmids, vectors derived from combinations of plasmids and phage DNA, viral DNA such as vaccinia, adenovirus, fowl pox virus and pseudorabies. However, any other plasmid or vector may be used so long as it is replicable and viable in the host.

The appropriate DNA sequence may be inserted into the vector by a variety of procedures. In general, the DNA sequence is inserted into appropriate restriction endonuclease sites by procedures known in the art. The polynucleotide sequence in the expression vector is operatively linked to an appropriate expression control sequence (i.e. promoter) to direct mRNA synthesis. Examples of such promoters include, but are not limited to, the LTR or the SV40 promoter, the E. coli lac or trp, the phage lambda P_{L} promoter and other promoters known to control expression of genes in prokaryotic or eukaryotic cells or their viruses. The expression vector also contains a ribosome binding site for translation initiation and a transcription terminator. The vector may also include appropriate sequences for amplifying expression. For example, the vector can contain enhancers, which are transcription-stimulating DNA sequences of viral origin, such as those derived form simian virus such as SV40, polyoma virus, bovine papilloma virus or Moloney sarcoma virus, or genomic, origin The vector preferably also contains an origin of replication. The vector can be constructed to contain an exogenous origin of replication or, such an origin of replication can be derived from SV40 or another viral source, or by the host cell chromosomal replication mechanism.

In addition, the vectors preferably contain a marker gene for selection of transfected host cells such as dihydrofolate reductase or antibiotics, such as G-418 (geneticin, a neomycin-derivative) or hygromycin, or genes which complement a genetic lesion of the host cells such as the absence of thymidine kinase, hypoxanthine phosphoribosyl transferase, dihydrofolate reductase, etc.

Suitable vectors for use in the present invention are known in the art. Any plasmid or vector can be used in the present invention as long as it is replicable and is viable in the host. Examples of vectors that can be used include those that are suitable for mammalian hosts and based on viral replication systems, such as simian virus 40 (SV40), Rous sarcoma virus (RSV), adenovirus 2, bovine papilloma virus (BPV), papovavirus BK mutant (BKV), or mouse and human cytomegalovirus (CVM).

In a further embodiment, the present invention relates to host cells containing the above described constructs. The host cell can be a higher eukaryotic cell, such as a mammalian cell, or a lower eukaryotic cell, such as a yeast cell, or the host cell can be a prokaryotic cell, such as a bacterial cell. Preferably, the host cells provide a suitable environment for the production of active antibodies, since the biosynthesis of functional tetrameric antibody molecules requires correct nascent polypeptide chain folding, glycosylation, and assembly. Example of suitable host cells, include mammalian cells, such as COS-7 cells, Bowes melanoma cells, Chinese hamster ovary (CHO) cells, embryonic lung cells L-132, and mammalian cells of lymphoid origin, such as myeloma or lymphoma cells. The host cells can be transfected with a vector containing a polynucleotide sequence encoding the H-chain alone, with a second vector encoding the light chain alone (such as by using two different vectors as discussed previously). Preferably, the host cells are transfected with two different vectors.

Introduction of the vectors into the host cell can be effected by calcium phosphate transfection, DEAE-Dextran mediated transfection or electroporation (L. David et al., Basic Methods in Molecular Biology 2^{nd} Edition, Appleton and Lang, Paramount Publishing, East Norwalk, Conn. (1994)).

In order to obtain the antibodies of the present invention, one or more polynucleotide sequences that encode for the light and heavy chain variable regions and light and heavy chain constant regions of the antibodies of the present invention should be incorporated into a vector. Polynucleotide sequences encoding the light and heavy chains of the antibodies of the present invention can be incorporated into one or multiple vectors and then incorporated into the host cells.

The antibodies of the present invention have a number of uses. The antibodies of the present invention can be used to identify and diagnose mammals that have a dysfunctional EPO receptor. Mammals that have a dysfunctional EPO receptor are characterized by disorders such as anemia. Preferably, the mammal being identified and diagnosed is a human. Additionally, the antibodies of the present invention can be used in the treatment of anemia in mammals suffering from aplasia resulting from the administration of EPO.

In yet another embodiment, the present invention relates to a pharmaceutical composition containing a therapeutically effective amount of the antibody of the present invention along with a suitable carrier or excipient. Suitable excipients include but are not limited to fillers such as sugars, including lactose, sucrose, mannitol, sorbitol, and the like, cellulose preparations such as, maize starch, wheat starch, rice starch, potato starch, gelatin, gum tragacanth, ethyl cellulose, hydroxypropylmethylcellulose, sodium carboxymethylcellulose, polyvinylpyrrolidone (PVP), and the like, as well as mixtures of any two or more. Optionally, disintegrating agents can be included, such as cross-linked polyvinyl pyrrolidone, agar, alginic acid or a salt thereof, such as sodium alginate and the like.

In addition to the excipients, the pharmaceutical composition can include one or more of the following, carrier proteins such as serum albumin, buffers, binding agents, sweeteners and other flavoring agents; coloring agents and polyethylene glycol.

Suitable routes of administration for the pharmaceutical composition include, but are not limited to, rectal, transdermal, vaginal, transmucosal or intestinal administration, parenteral delivery, including intramuscular, subcutaneous, intramedullary injections, as well as intrathecal, direct intraventricular, intravenous, intraperitoneal, intranasal, intraocular, and the like.

In yet another embodiment, the present invention relates to a method of modulating the endogenous activity of the EPO receptor in a mammal, such as, activating or increasing the activity of the EPO receptor in such a mammal. The method involves the step of administering to a mammal, such as a human suffering from a dysfunctional EPO receptor, a therapeutically effective amount of the antibodies of the present invention. Additionally, the present invention further relates to a method of treating pure red cell aplasia induced by neutralizing anti-erythropoietin antibodies. The method is useful for treating mammals suffering from red cell aplasia resulting from the administration of recombinant EPO (See, Casadevall, N., “Pure Red-Cell Aplasia and Anti-erythropoietin Antibodies in Patients Treated with Recombinant Erythropoietin,” N. Engl. J. Med., 346 (7):469-75 (Feb. 14, 2002); Casadevall, N., “Antibodies Against rHuEPO: Native and Recombinant,” Nephrol. Bial. Transplant, 17 Suppl. 5:42-47 (2002)). The method involves the step of administering to a mammal suffering from said apalsia and in need of treatment a therapeutically effective amount of the antibodies of the present invention.

As used herein, the term “therapeutically effective amount” means an amount that produces the effects for which it is administered. The exact dose will be ascertainable by one skilled in the art. As known in the art, adjustments based on age, body weight, sex, diet, time of administration, drug interaction and severity of condition may be necessary and will be ascertainable with routine experimentation by those skilled in the art.

Suitable routes of administration for the antibodies of the present invention include, but are not limited to, oral, rectal, transdermal, vaginal, transmucosal or intestinal administration, parenteral delivery, including intramuscular, subcutaneous, intramedullary injections, as well as intrathecal, direct intraventricular, intravenous, intraperitoneal, intranasal, intraocular, and the like.

By way of example, and not of limitation, examples of the present invention shall now be given.

### EXAMPLE 1

Generation of Human Erythropoietin Receptor Antibodies

Antigen Preparation. The antigen used for immunization of XenoMouse® animals was coupled to a universal T-cell epitope (TCE) (J.Immunol., 148(5):1499 (1992)) using two different methods. A mixture containing an equal amount of each was used as the immunogen.

1) 2.3 mg of Dithiothreitol (DTT), and 200 mcg of cysteine coupled TCE (J.Immunol., 148(5):1499 (1992)) are mixed at room temperature for 30 minutes. DTT is removed by centrifugation through a Sephadex G10 (Pharmacia, Upsala, Sweden) chromatography column. The reduced cysteine coupled TCE is added to 200 mcg soluble extracellular domain of human EpoR (R&D Systems, Minneapolis, Minn.) re-suspended in Phosphate Buffered Saline (PBS) (8.1 mM Na₂HPO₄, 1.6 mM NaH₂PO₄, 136 mM NaCl, 2.6 mM KCl, pH 7.4) and 33 mcg of Sulfosuccinimidyl 4-(N-maleimidomethyl)-cyclohexane-1-carboxylate (sulfo SMCC), and mixed 4° C. over night. Un-reacted EpoR was removed by centrifugation through a 10 KDa cut off Centricon column (Millipore, Bedford, Mass.).

2) The soluble extracellular domain (200 mcg) of human EpoR (R&D Systems, Minneapolis, Minn.) was re-suspended in PBS and mixed with 4 mcg of TCE-BPA (p-Benzoyl Phenylalanine) and incubated under UV light (362 nM) at room temperature for 45 minutes. The un-reacted EpoR was removed by centrifugation through a 10 KDa cut off Centricon column (Millipore, Bedford, Mass.).

Immunization of animals. Monoclonal antibodies of the invention, including Ab12 and Ab198 (also referred to herein as AB-ABT2-XG2-012 and AB-ABT2-XG2-198, respectively) were developed by immunizing XenoMouse® mice (XenoMouse® XG2, Abgenix, Inc., Fremont, Calif. and Vancouver, BC) with soluble EpoR coupled to a TCE as described above. The initial immunization was with 20 mcg of antigen and mixed 1:1 v/v with Complete Freund's Adjuvant (CFA) (Sigma, St Louis, Mo.) per mouse. The subsequent immunizations were with 20 mcg of antigen mixed 1:1 v/v with incomplete Freund's (IFA). In particular, each animal was immunized at the base of tail and by intraperitoneal injection on days 0, 14, 28 and 42.

Biotinylation of EpoR. 300 mcg of EpoR (Abbott CHO cell derived ref.# RB69084:4) was re-suspended in 990 mcL of PBS pH 8.6 and added to 100 mcg of biotin-NHS (Pierce, Rockford, Ill.) dissolved in DMSO (Dimethyl Sulfoxide) incubated for forty minutes at room temperature (RT). Free biotin and buffer was removed by centrifugation through a 5 kDa Centricon column with several washes with PBS pH 7.4 and re-suspended in an appropriate volume to a final concentration was 600 mcg/mL.

Selection of animals for harvest. Anti-EpoR antibody titers were determined by ELISA. 0.7 mcg/ml biotin EpoR (described above) was coated onto streptavadin plates (Sigma, St Louis, Mo.) at room temperature for 1 hour. The solution containing unbound biotin EpoR was removed and all plates were washed five times with dH₂O. XenoMouse® sera from the EpoR immunized animals, or naive XenMouse® animals, were titrated in 2% milk/PBS at a 1:2 dilution in duplicate from a 1:100 initial dilution. The last well was left blank, and plates were washed five times with dH₂O. A goat anti-human IgG Fc-specific horseradish peroxidase (HRP)(Pierce, Rockford, Ill.) conjugated antibody was added at a final concentration of 1 mcg/mL for 1 hour at room temperature. The plates were washed five times with dH₂O. The plates were developed with the addition of TMB chromogenic substrate (KPL, Gaithersburg, Md.) for 30 minutes and the ELISA was stopped by the addition of 1 M phosphoric acid. The specific titers obtained from XenoMouse® animals were determined from the optical density at 450 nm and are shown in Table 1. The titer represents the reciprocal dilution of the serum and therefore the higher the number the greater the humoral immune response to EpoR.

TABLE 1

| Mouse I.D. | Titer |
|---|---|
| 11 | 1600 |
| 12 | 12800 |
| 13 | 51200 |
| 14 | 102400 |
| 15 | 102400 |
| 16 | 0 |
| 17 | 102400 |
| 18 | 3200 |
| 19 | 102400 |
| 20 | 2560 |

XenoMouse® animal 14 was selected for harvest based on the serology data in Table 1.

Culture and selection of B cells. B cells from the harvested animals were cultured and those secreting EpoR-specific antibodies were isolated essentially as described in Babcook et al., Proc. Natl. Acad. Sci. USA, 93:7843-7848 (1996). ELISA, performed as described above for sera titers, was used to identify EpoR-specific wells. Fifty plates cultured at 500 cells/well were screened on biotin EpoR to identify the antigen-specific wells. The data as shown in Table 2 demonstrated the presence of 701 wells with ODs significantly over background (0.05).

TABLE 2

| Optical Density | Number of Positives |
|---|---|
| 0.1 | 701 |
| 0.2 | 273 |
| 0.3 | 163 |
| 0.4 | 130 |
| 0.5 | 102 |
| 0.6 | 91 |
| 0.7 | 76 |
| 0.8 | 70 |
| 0.9 | 67 |
| 1.0 | 65 |
| 2.0 | 25 |
| 3.0 | 7 |

These data indicated a very low frequency of hits and indicated that the wells were monoclonal for antigen-specificity. These 701 positive wells were rescreened on biotin EpoR and 137 wells (shown in bold in Table 3 below) were found to repeat as real antigen-specific wells with ODs significantly over background (0.05).

TABLE 3

| Optical Density | Number of Positives |
|---|---|
| 0.1 | 207 |
| 0.15 | 137 |
| 0.2 | 110 |
| 0.3 | 94 |
| 0.4 | 85 |
| 0.5 | 79 |
| 0.6 | 71 |
| 0.7 | 63 |
| 0.8 | 57 |
| 0.9 | 53 |
| 1.0 | 50 |
| 2.0 | 32 |
| 3.0 | 13 |

Agonist activity assay. Proliferation of an Epo responsive cell line was used as the basis for the agonist screen. These 137 wells were then screened for agonist activity using the human erythroleukemia cell line UT-7/Epo (Abbott ref#.RB29454-174). 12.5 mcL of supernatant were added to 1×105 cells per well in RPMI 1640 (10% FCS) to a final volume of 50 mcL in a half area 96 well plate. The well size is half the area of a typical 96 well plate. Proliferation was identified visually and compared to cells in media containing a titration of human Epo or no Epo as a base line control. Eleven wells with proliferation activity were identified.

EpoR-specific Hemolytic Plaque Assay. A number of specialized reagents are needed to conduct the assay. These reagents were prepared as follows.

Biotinylation of Sheep red blood cells (SRBC). SRBC are stored in RPMI media as a 25% stock. A 250 ul SRBC packed-cell pellet was obtained by aliquoting 1.0 ml of the stock into a 15-ml falcon tube, spinning down the cells and removing the supernatant. The cell pellet was then re-suspended in 4.75 ml PBS at pH 8.6 in a 50 ml tube. In a separate 50 ml tube, 2.5 mg of Sulfo-NHS biotin was added to 45 ml of PBS at pH 8.6. Once the biotin had completely dissolved, 5 ml of SRBCs were added and the tube rotated at RT for 1 hour. The SRBCs were centrifuged at 3000 g for 5 min, the supernatant drawn off and 25 mls PBS at pH 7.4 as a wash. The wash cycle was repeated 3 times, then 4.75 ml immune cell media (RPMI 1640 with 10% FCS) was added to the 250 ul biotinylated-SRBC (B-SRBC) pellet to gently re-suspend the B-SRBC (5% B-SRBC stock). Stock was stored at 4° C. until needed.

Streptavidin (SA) coating of B-SRBC. One ml of the 5% B-SRBC stock was transferred into to a fresh eppendorf tube. The B-SRBC cells were pelleted with a pulse spin at 8000 rpm (6800 rcf) in a microfuge, the supernatant drawn off, the pellet re-suspended in 1.0 ml PBS at pH 7.4, and the centrifugation repeated. The wash cycle was repeated 2 times, then the B-SRBC pellet was resuspended in 1.0 ml of PBS at pH 7.4 to give a final concentration of 5% (v/v). 10 ul of a 10 mg/ml streptavidin (CalBiochem, San Diego, Calif.) stock solution was added and the tube mixed and rotated at RT for 20min. The washing steps were repeated and the SA-SRBC were re-suspended in 1 ml PBS pH 7.4 (5% (v/v)).

EpoR coating of SA-SRBC. The SA-SRBC were coated with biotinylated EpoR at 10 ug/ml, the mixed and rotated at RT for 20 min. The SRBC were washed twice with 1.0 ml of PBS at pH 7.4 as above. The EpoR-coated SRBC were re-suspended in RPMI (+10% FCS) to a final concentration of 5% (v/v).

Determination of the quality of EpoR-SRBC by immunofluorescence (IF). 10 ul of 5% SA-SRBC and 10 ul of 5% PTH-coated SRBC were each added to separate fresh 1.5 ml eppendorf tube containing 40 ul of PBS. The murine antiEpoR antibody (R&D Systems Cat.# MAB307) was added to each sample of SRBCs at 20 ug/ml. The tubes were rotated at RT for 25 min, and the cells were then washed three times with 100 ul of PBS. The cells were re-suspended in 50 ul of PBS and incubated with 40 mcg/mL Gt-anti mouse IgG Fc antibody conjugated to Alexa488 (Molecular Probes, Eugene, Oreg.). The tubes were rotated at RT for 25 min, and then washed with 100 ul PBS and the cells re-suspended in 10 ul PBS. 10 ul of the stained cells were spotted onto a clean glass microscope slide, covered with a glass coverslip, observed under fluorescent light, and scored on an arbitrary scale of 0-4.

Preparation of plasma cells. The contents of a single microculture well identified by the previous assays as containing a B cell clone secreting the immunoglobulin of interest were harvested. Using a 100-1000 ul pipettman, the contents of the well were recovered by adding 37C RPMI (+10% FCS). The cells were re-suspended by pipetting and then transfered to a fresh 1.5 ml eppendorf tube (final vol. approx 500-700 ul). The cells were centrifuged in a microfuge at 1500 rpm (240 rcf) for 2 minutes at room temperature, then the tube rotated 180 degrees and spun again for 2 minutes at 1500 rpm. The freeze media was drawn off and the immune cells resuspended in 100 ul RPMI (10% FCS), then centrifuged. This washing with RPMI (10% FCS) was repeated and the cells re-suspended in 60 ul RPMI (FCS) and stored on ice until ready to use.

Plaque assay. Glass slides (2×3 inch) were prepared in advance with silicone edges and allowed to cure overnight at RT. Before use the slides were treated with approx. 5 ul of SigmaCoat (Sigma, Oakville, ON) wiped evenly over glass surface, allowed to dry and then wiped vigorously. To a 60 ul sample of cells was added 60 ul each of EpoR-coated SRBC (5% v/v stock), 4× guina pig complement (Sigma, Oakville, ON) stock prepared in RPMI with 10% FCS, and 4× enhancing sera stock (1:900 in RPMI with 10% FCS). The mixture (3-5 ul) was spotted onto the prepared slides and the spots covered with undiluted paraffin oil. The slides were incubated at 37° C. for a minimum of 45 minutes.

Plaque assay results. The coating was determined qualitatively by immunofluorescent microscopy to be very high (4/4) using MAB307 to detect coating compared to a secondary detection reagent alone (0/4). There was no signal detected using the MAB307 antibody on red blood cells that were only coated with streptavidin (0/4). These red blood cells were then used to identify antigen-specific plasma cells from the fourteen wells identified in Table 4. After micromanipulation to rescue the antigen-specific plasma cells, the genes encoding the variable region genes were rescued by RT-PCR on a single plasma cell.

TABLE 4

| Plate ID | Single Cell numbers |
|---|---|
| 11G10 | ABT2-SCX-251-260 |
| 21D1 | ABT2-SCX-54 |
| 25C3 | ABT2-SCX-134-144 |
| 29G8 | ABT2-SCX-1-11 |
| 33G8 | ABT2-SCX-12-18 |
| 37A11 | ABT2-SCX-19-44 |
| 43H12 | ABT2-SCX-185-201, 233-239 |
| 16F7 | ABT2-SCX-267-278 |
| 24C3 | ABT2-SCX-55-77 |
| 24F8 | ABT2-SCX-82-102 |
| 34D4 | ABT2-SCX-145-168 |

Expression. After isolation of the single plasma cells, mRNA was extracted and reverse transcriptase PCR was conducted to generate CDNA. The cDNA encoding the variable heavy and light chains was specifically amplified using polymerase chain reaction. The variable heavy chain region was cloned into an IgG2 expression vector. This vector was generated by cloning the constant domain of human IgG2 into the multiple cloning site of pcDNA3.1+/Hygro (Invitrogen, Burlington, ON). The variable light chain region was cloned into an IgK expression vector. This vector was generated by cloning the constant domain of human IgK into the multiple cloning site of pcDNA3.1+/Neo (Invitrogen, Burlington, ON). The appropriate pairs of heavy chain and the light chain expression vectors were then co-lipofected into a 60 mm dish of 70% confluent human embryonal kidney 293 cells and the transfected cells were left to secrete a recombinant antibody for 24 hours. The supernatant (3 mL) was harvested from the HEK 293 cells and the secretion of an intact antibody (AB-ABT2-XG2-012 and AB-ABT2-XG2-198) was demonstrated with a sandwich ELISA to specifically detect human IgG (Table 5, fourth column). The specificity of AB-ABT2-XG2-012 and AB-ABT2-XG2-198 was assessed through binding of the recombinant antibody to biotinylated EpoR using ELISA (Table 5, fifth column).

TABLE 5

| Well ID | Single cell number | Secretion | Binding |
|---|---|---|---|
| 11G10 | ABT2-SCX-254 | 1:4 | 1:8 |
| 21D1 | ABT2-SCX-054 | >1:64 | >1:64 |
| 25C3 | ABT2-SCX-135 | 1:4 | 1:4 |
| 29G8 | ABT2-SCX-003 | >1:64 | >1:64 |
| 33G8 | ABT2-SCX-012 | >1:64 | >1:64 |
| 37A11 | ABT2-SCX-022 | >1:64 | >1:64 |
| 43H12 | ABT2-SCX-198 | >1:64 | >1:64 |
| 16F7 | ABT2-SCX-267 | >1:64 | >1:64 |
| 24C3 | ABT2-SCX-060 | >1:64 | >1:64 |
| 24F8 | ABT2-SCX-102 | >1:64 | >1:64 |
| 34D4 | ABT2-SCX-145 | >1:64 | >1:64 |

The ELISA for antigen specific antibody secretion was performed as follows. Control plates were coated with 2 mg/mL Goat anti-human IgG H+L O/N. For the binding plates, biotin-EpoR (0.7 mcg/mL) was coated onto streptavadin 96 well plates (Sigma, St Louis, Mo.) for one hour at room temperature. The plates were washed five times with dH₂O. Recombinant antibodies were titrated 1:2 for 7 wells from the undiluted minilipofection supernatant. The plates were washed five times with dH₂O. A goat anti-human IgG Fc-specific HRP-conjugated antibody was added at a final concentration of 1 ug/mL for 1 hour at RT for the secretion and the binding ELISA. The plates were washed five times with dH₂O. The plates were developed with the addition of TMB chromogenic substrate (KPL, Gaithersburg, Md.) for 30 minutes and the ELISA was stopped by the addition of 1 M phosphoric acid. Each ELISA plate was analyzed to determine the optical density of each well at 450 nm.

Purification of AB-ABT2-XG2-012 and AB-ABT2-XG2-198. For larger scale production, the heavy and light chain expression vectors (2.5 ug of each chain/dish) were lipofected into ten 100 mm dishes that were 70% confluent with HEK 293 cells. The transfected cells were incubated at 37° C. for 4 days, the supernatant (6 mL) was harvested and replaced with 6 mL of fresh media. At day 7, the supernatant was removed and pooled with the initial harvest (120 mL total from 10 plates). The ABT2-XG2-012 and ABT2-XG2-198 antibody were purified from the supernatant using a Protein-A Sepharose (Amersham Biosciences, Piscataway, N.J.) affinity chromatography (1 mL). The antibody was eluted from the Protein-A column with 500 mcL of 0.1 M Glycine pH 2.5. The eluate was dialysed in PBS pH 7.4 and filter sterilized. The antibody was analyzed by non-reducing SDS-PAGE to assess purity and yield.

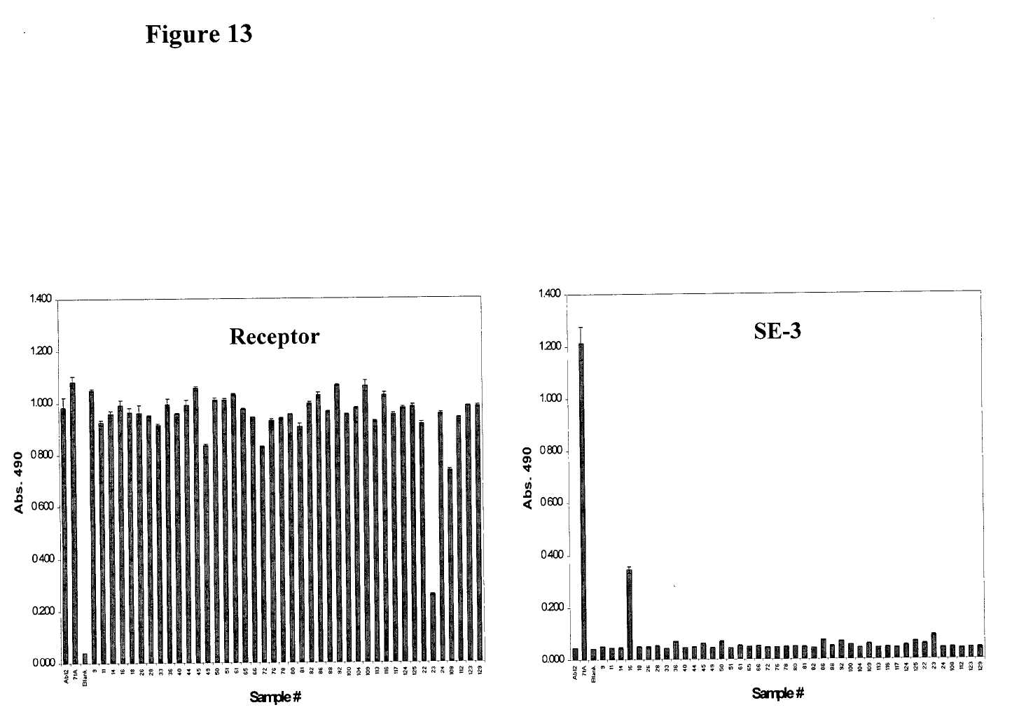

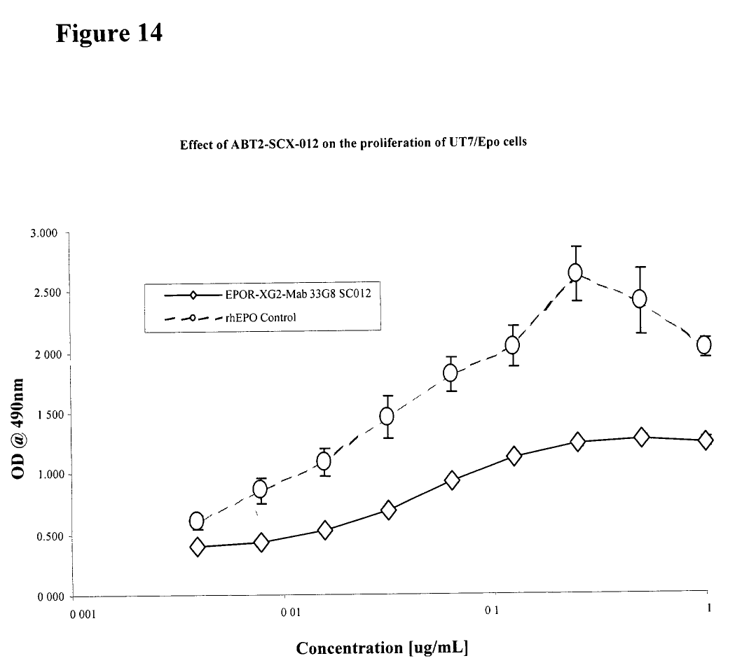

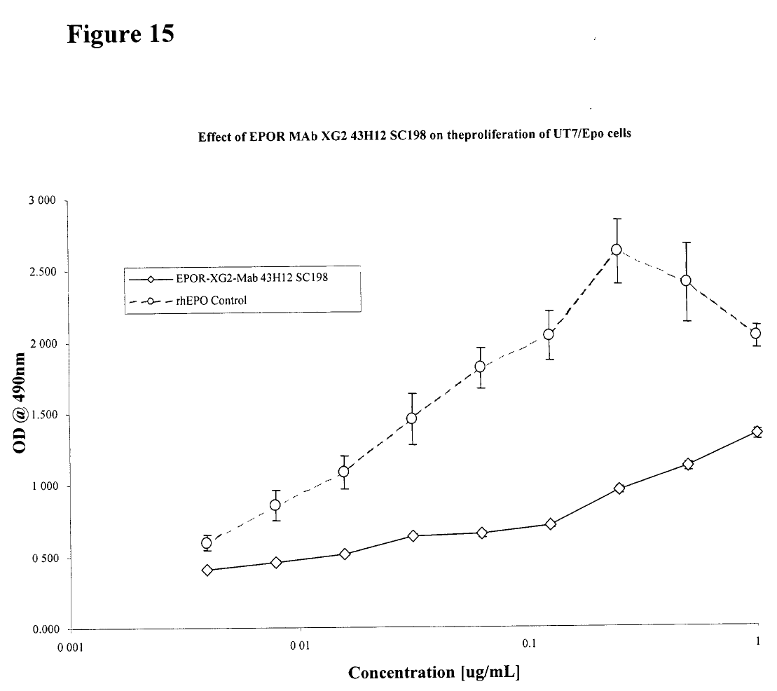

Agonist activity of recombinant antibodies. The ability of these recombinant antibodies to stimulate the proliferation of Epo responsive cells was examined using the UT-7/Epo cells with proliferation quantitated by MTS reagent (Promega, Madison, Wis.) measured at 490 nm as described in the Agonist Activity Assay above. ABT2-SCX-012 and ABT2-SCX-198 induced proliferation in comparison to cells in media without antibody and are shown below (FIGS. 14 and 15 respectively).

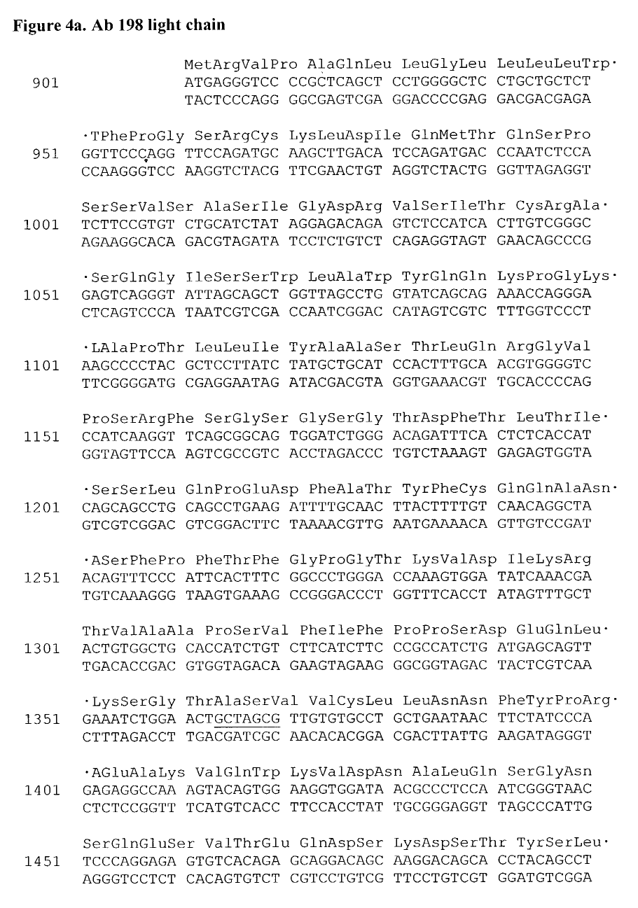

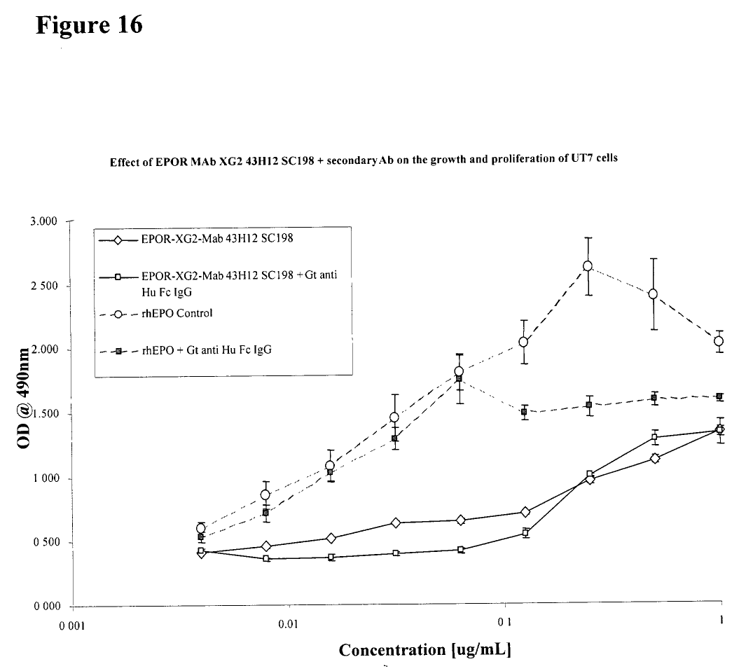

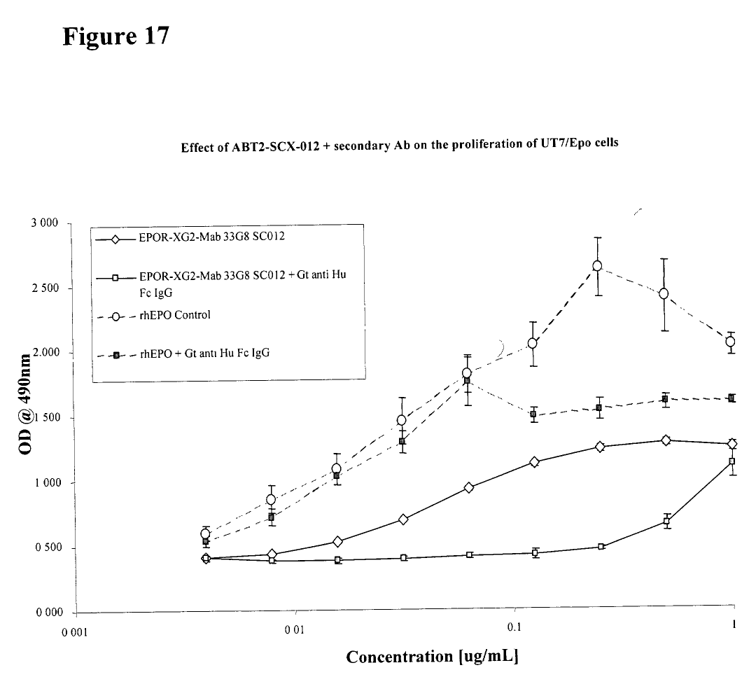

Effect of anti-Human Fc. It is possible that the agonist activity of ABT2-SCX-012 and ABT2-SCX-198 are due to self-aggregation. In order to address this issue we induced aggregation by the addition of an anti-human Fc secondary antibody and the effect on the agonist activity of ABT2-SCX-012 and ABT2-SCX-198 was determined using the UT-7/Epo cells. As shown below the addition of a secondary antibody had no effect on the activity of ABT2-SCX-198 (FIG. 16) and inhibited the activity of ABT2-SCX-012 (FIG. 4 17).

Since the addition of secondary Ab inhibited the activity of ABT2-SCX-012 we concluded that aggregation of this antibody interferes with it's activity and thus it is unlikely that ABT2-SCX-012 has agonist activity due to aggregation. However, the results of ABT2-SCX-198 are more difficult to interpret. The lack of an effect could suggest that ABT2-SCX-198 is fully aggregated and thus the addition of secondary Ab has no further effects on its activity. Alternatively, the lack of effect suggests the activity of ABT2-SCX-198 is not perturbed by the conformational restrictions applied by a secondary antibody.

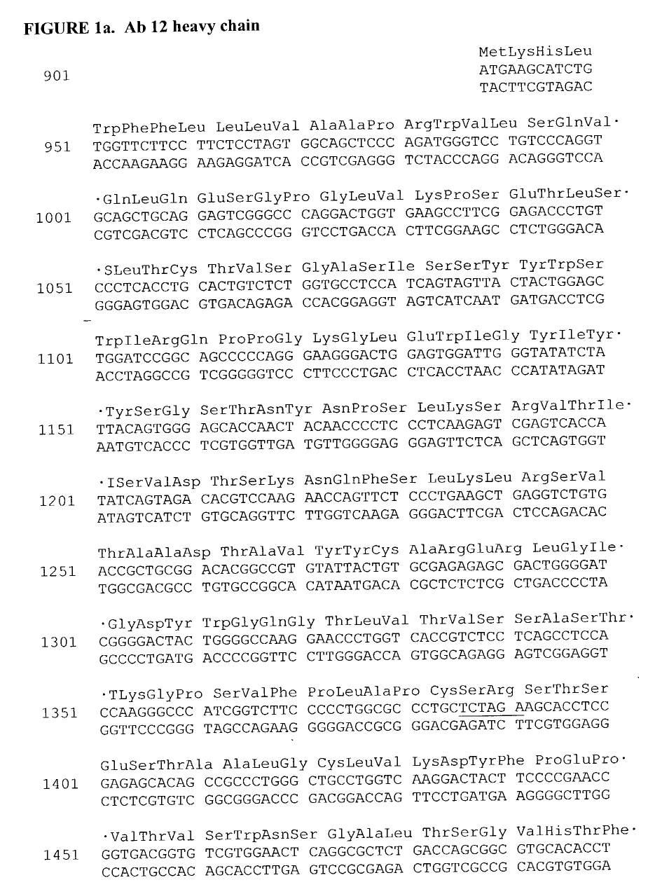

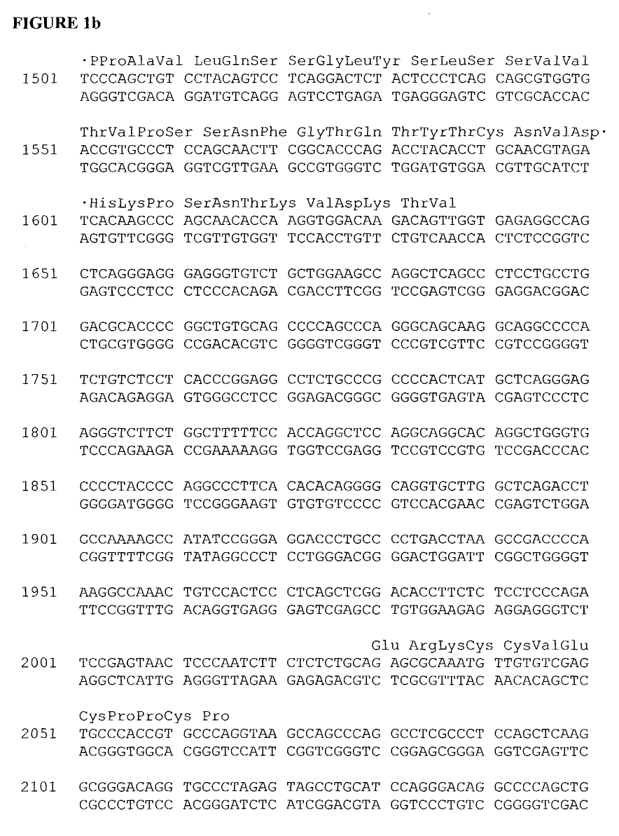

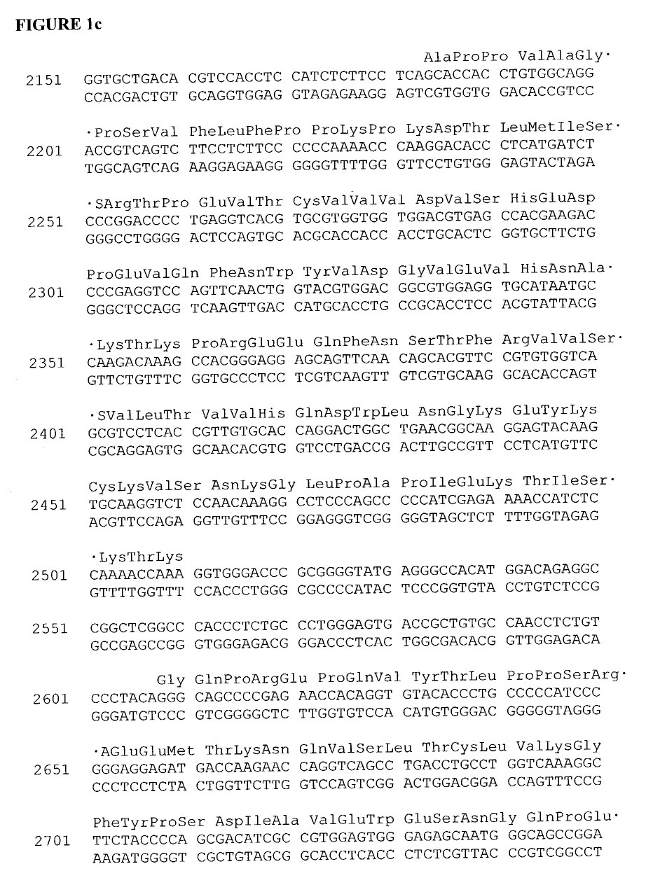

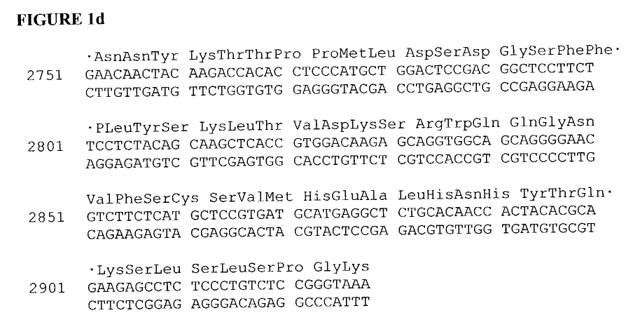

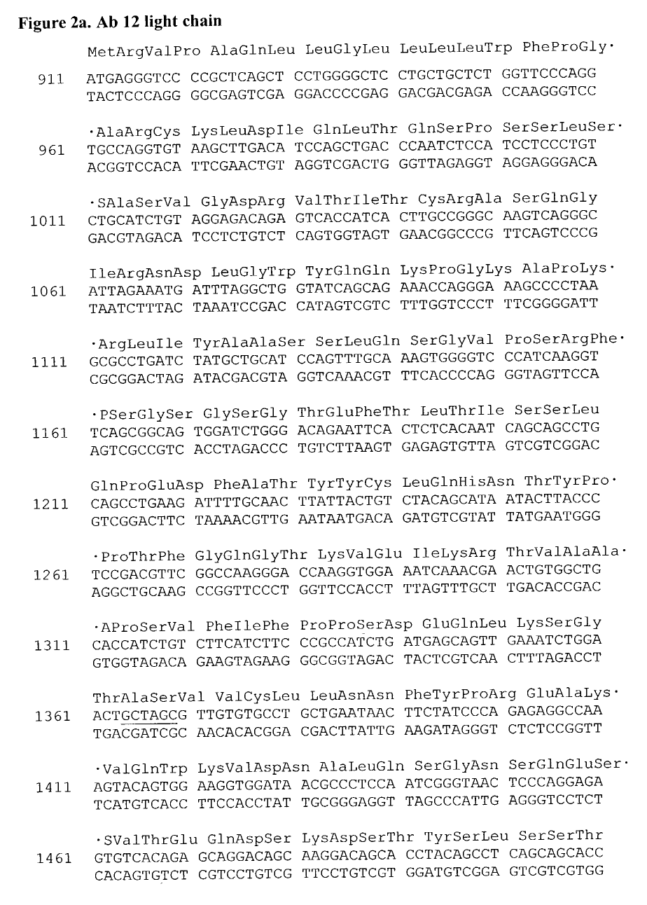

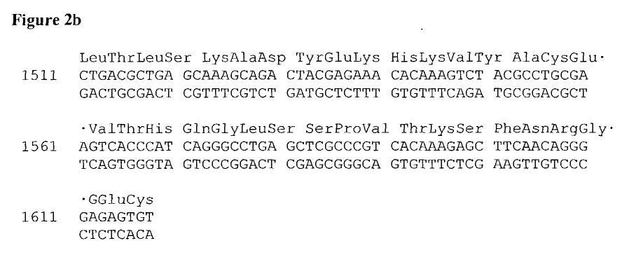

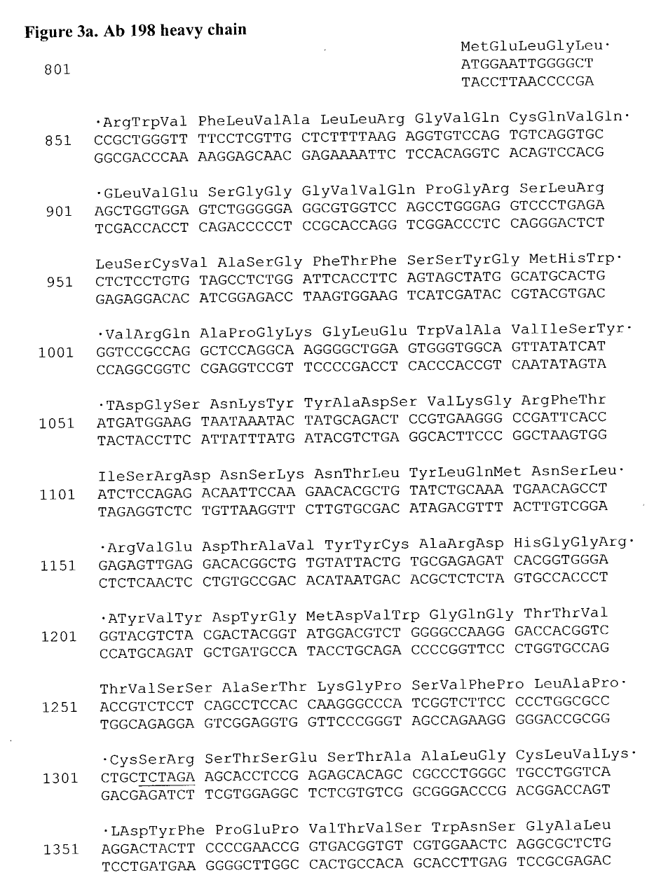

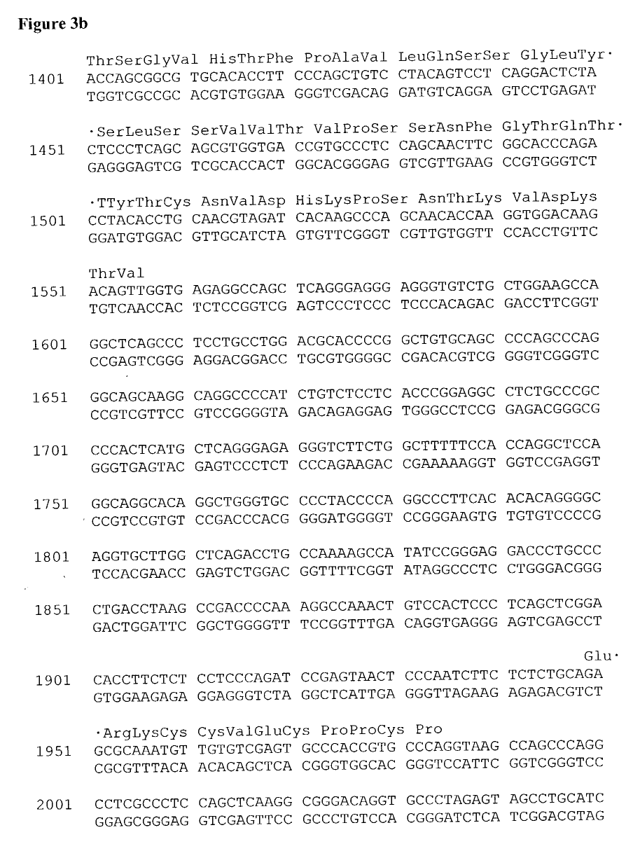

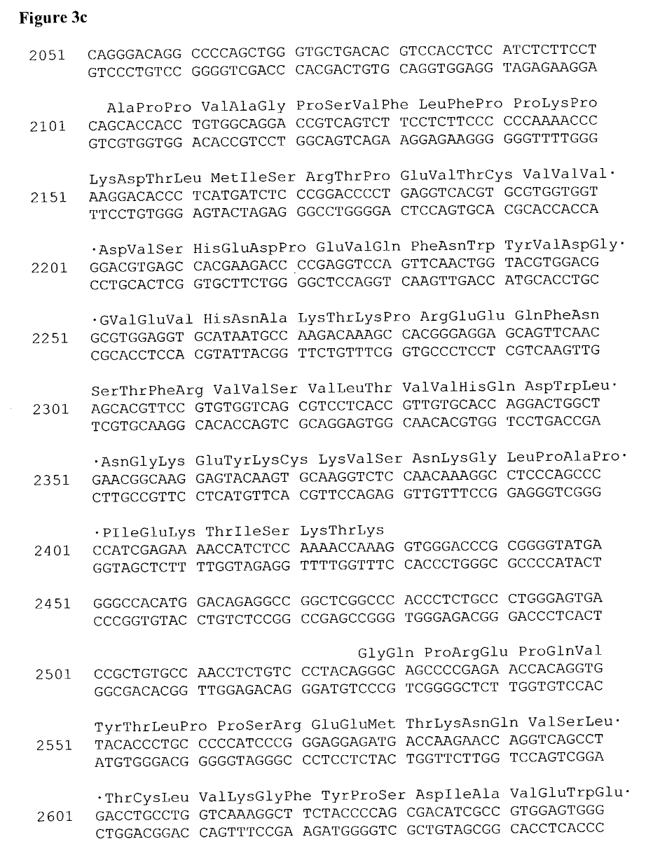

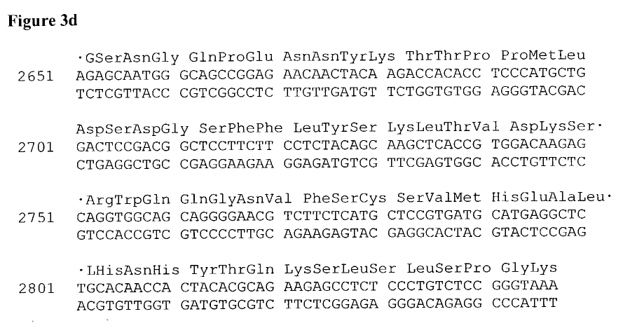

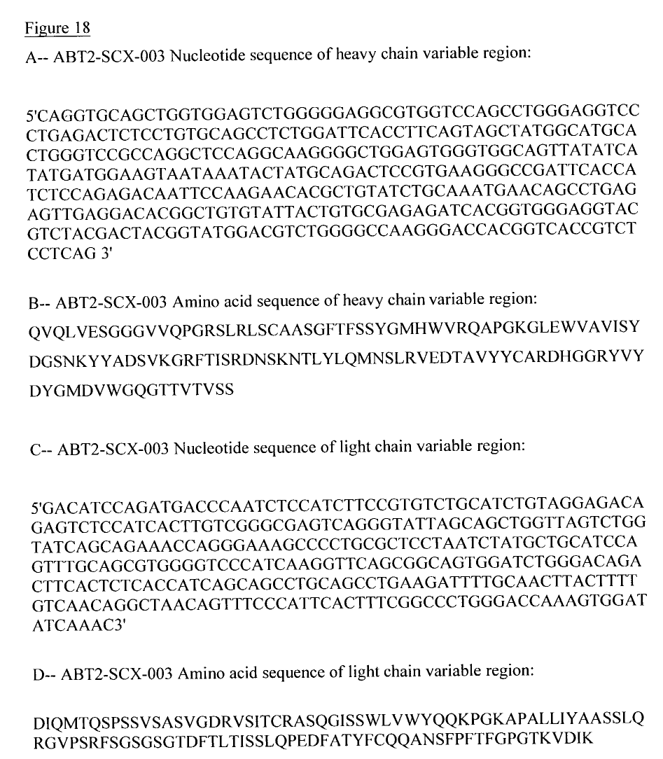

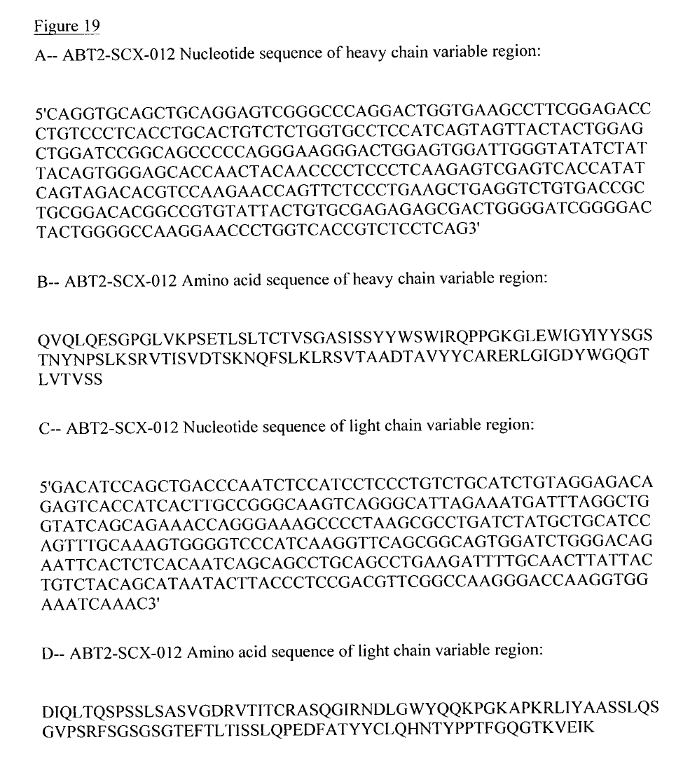

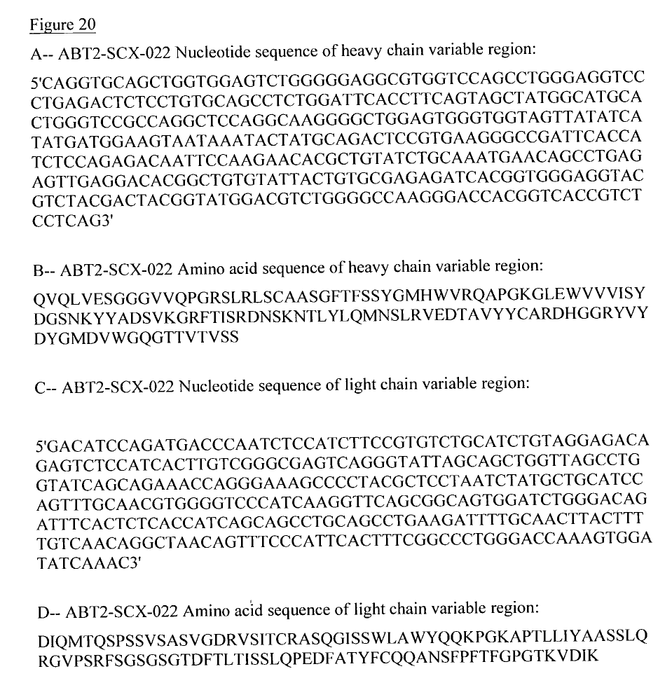

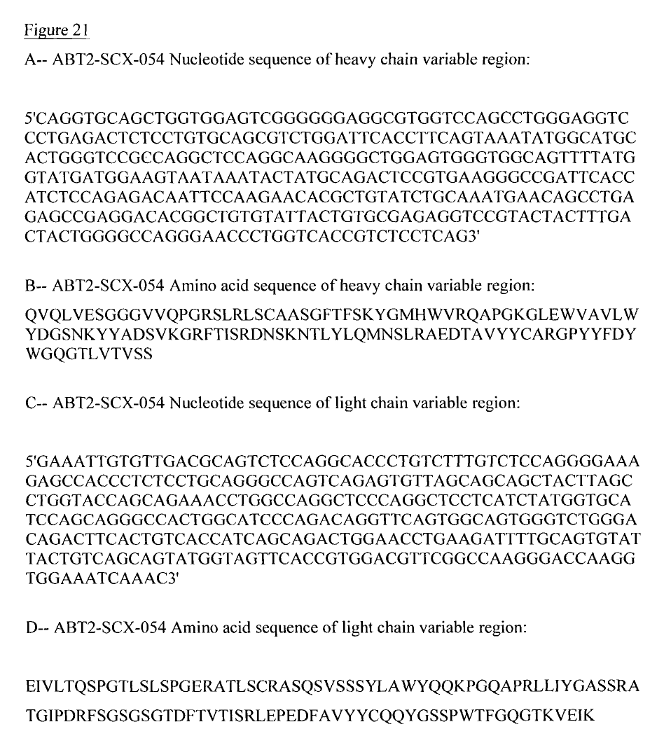

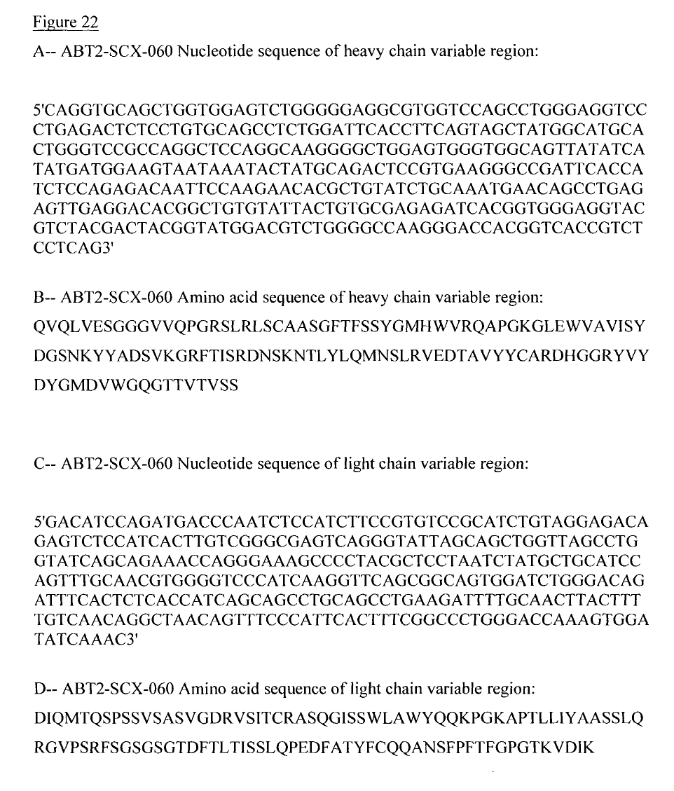

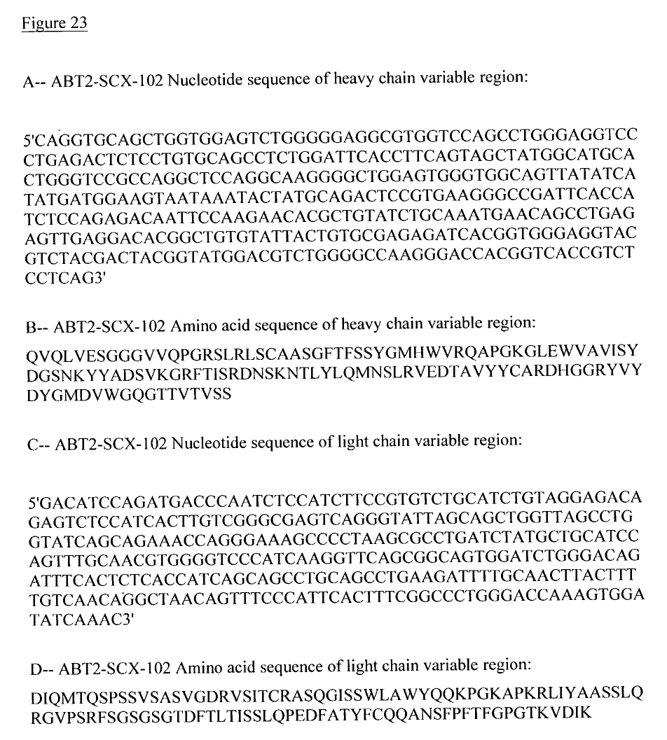

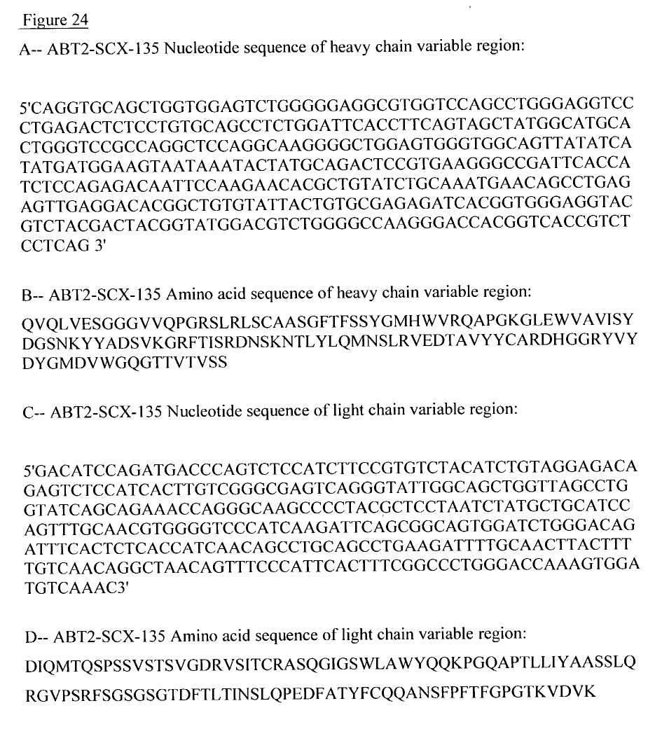

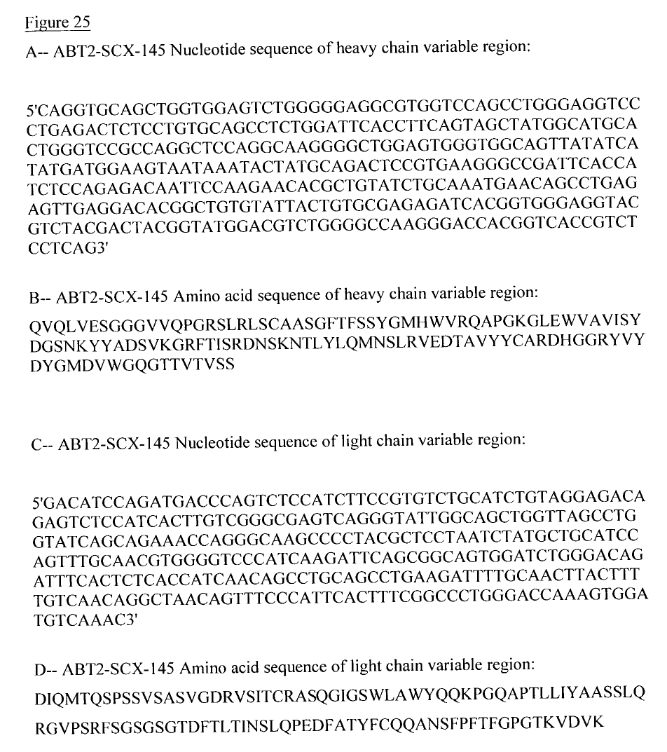

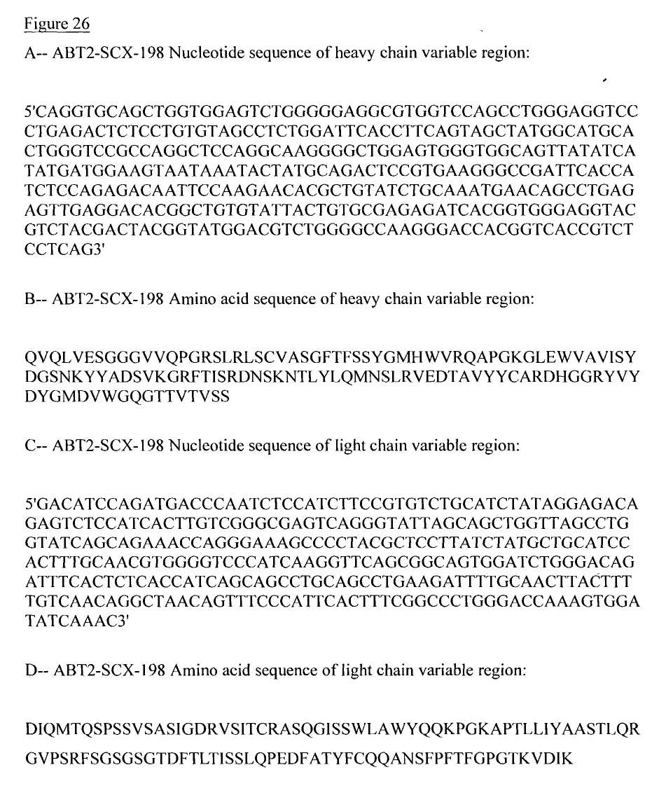

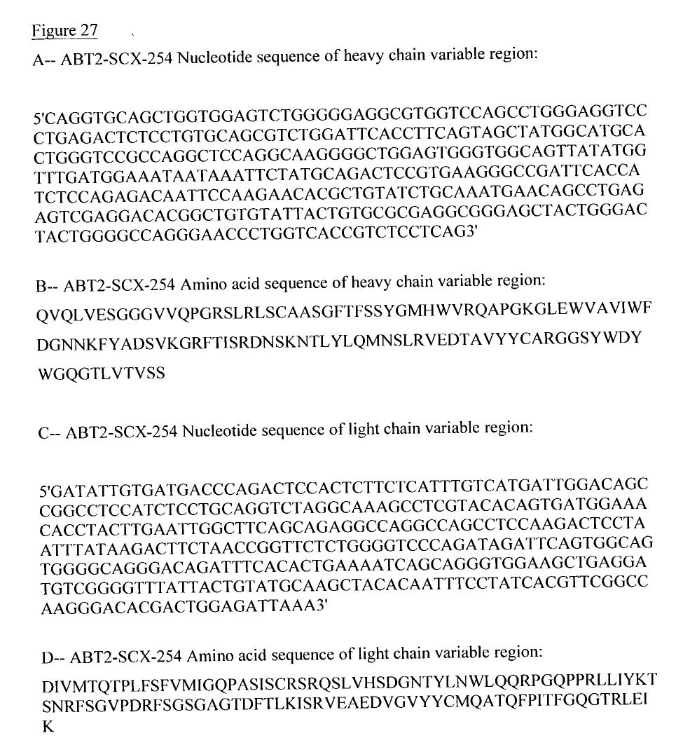

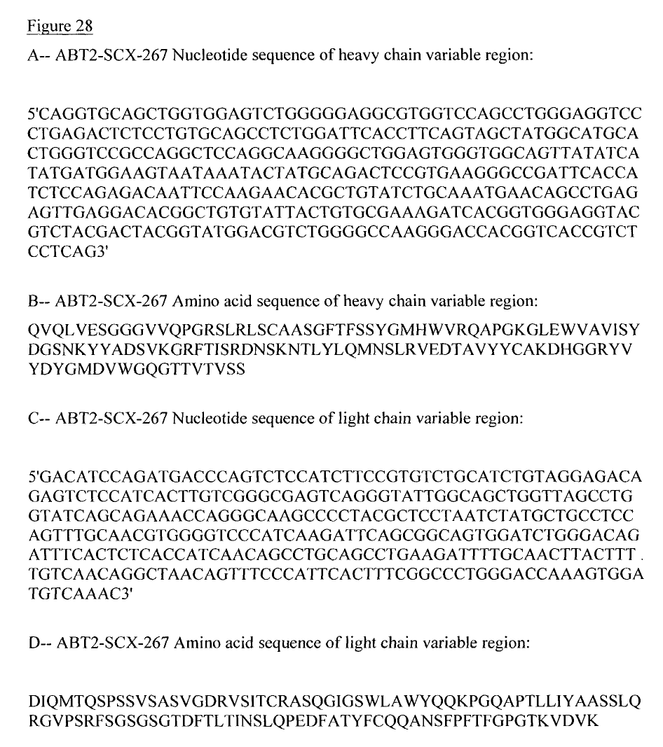

Sequence analysis of ABT2-SCX-012 and ABT2-SCX-198 The variable heavy chains and the variable light chains for antibodies ABT2-SCX-012 and ABT2-SCX-198 were sequenced to determine their DNA sequences. The complete sequence information for the anti-EpoR antibodies shown in FIGS. 1, 2, and 18-30 with nucleotide and amino acid sequences for each variable region of the heavy chain gamma and kappa light chains. FIGS. 1 and 2 provide full-length sequences, including the constant regions.

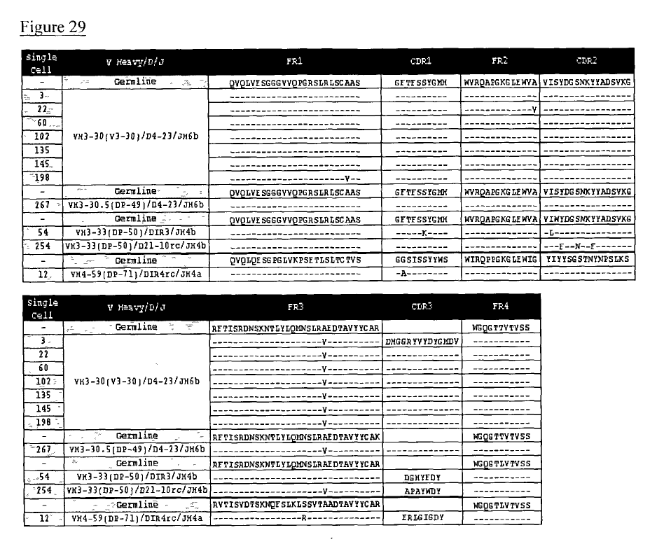

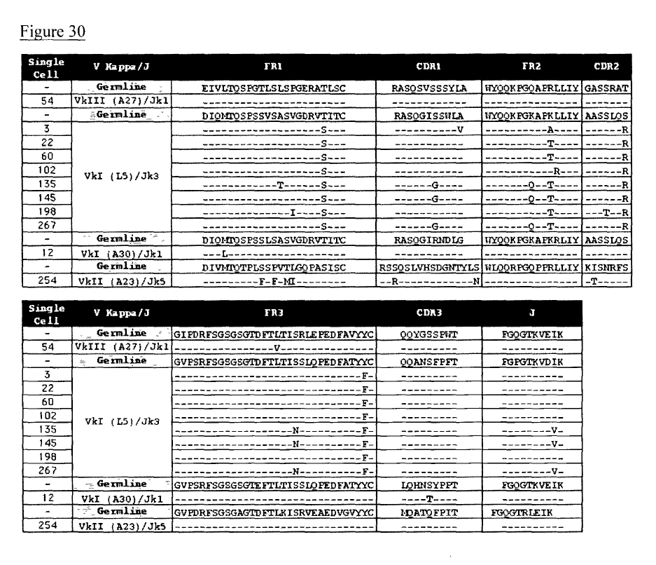

The variable heavy sequences were analyzed to determine the VH family, the D-region sequence and the J-region sequence. The sequences were then translated to determine the primary amino acid sequence (FIG. 29) and compared to the germline VH, D and J-region sequences to assess somatic hypermutations. The primary amino acid sequences of all the anti-EpoR antibody gamma chains are shown in FIG. 16. The germline sequences are shown above and the mutations are indicated with the new amino acid sequence. Unaltered amino acids are indicated with a dash (-). The light chain was analyzed similarly to determine the V and the J-regions and to identify any somatic mutations from germline kappa sequences (FIG. 30). The heavy chain of ABT2-SCX-012 was shown to utilize the VH 4-59 (DP-71), DIR4rc and the JH4a gene segments, while the light chain was shown to use the VkI (A30) and the Jk1 gene segments. The heavy chain of ABT2-SCX-198 was shown to utilize the VH 3-30 (V3-30), D4-23 and the JH6b gene segments, while the light chain was shown to use the VkI (L5) and the Jk3 gene segments.

### EXAMPLE 2

Competition of Ab12 with ¹²⁵I-Labeled EPO for Binding CHO Cells Expressing Recombinant EPO Receptor

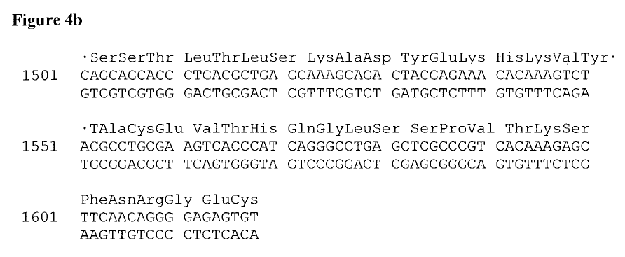

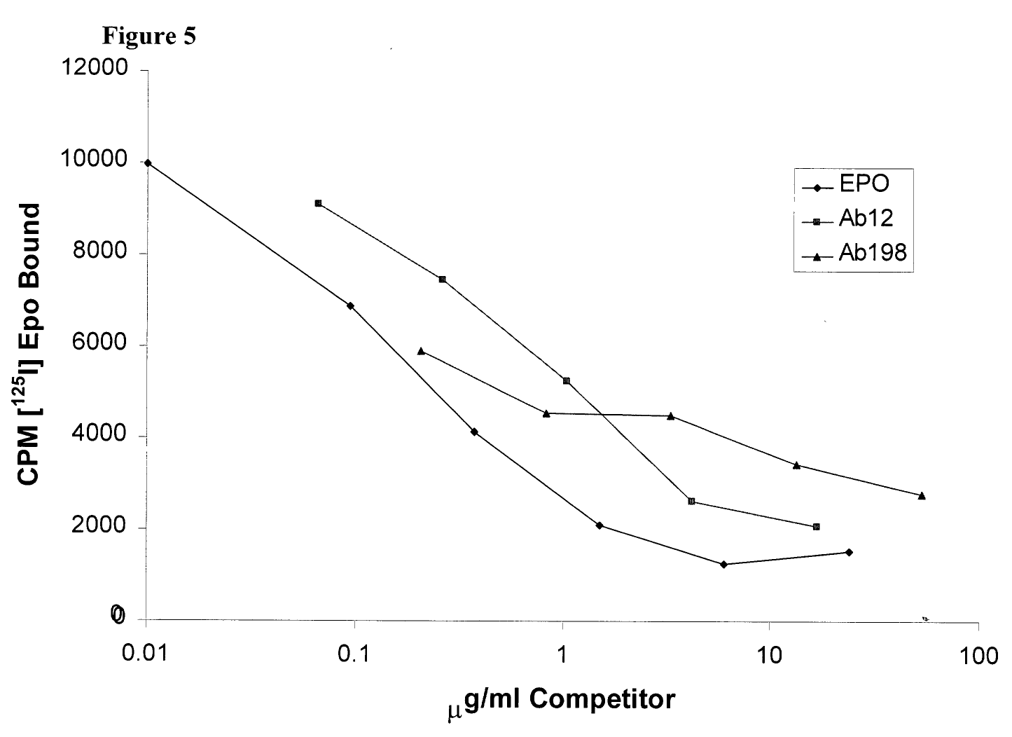

CHO cells expressing the full length recombinant human EPO receptor were plated at 5×10⁵ cells/well in 24 well plates 72 hours prior to the assay. On the day of the assay, 95 ul of Ab12, Ab198, or EPO at indicated concentrations (shown in FIG. 5) diluted in RPMI 1640, 0.5% BSA, 1 mM Na N₃ and 5 ul (6 ng) of ¹²⁵I-EPO (Amersham Cat. #IM178, Arlington Heights, Ill. 486 ci/mM) were added to the wells. After incubating at 37° C. for 1.5 hours, the wells were washed three times with cold HBSS and harvested using 0.5 ml 0.1N NaOH. Samples were counted in a Micromedic ME Plus gamma counter. The results are shown in FIG. 5. Specifically, the results show that Abs 12 and 198 competed with EPO for binding to the erythropoietin receptor.

### EXAMPLE 3

Biacore Studies

The studies described below were performed on a Biacore 2000 utilizing the Biacontrol software version 3.1. (Biacore, Uppsala, Sweden). Binding analyses were performed with antibody immobilized directly to the chip surface and followed by injection of varying receptor concentrations.

Immobilization of Antibody

Immobilizations of antibody were performed using the default immobilization program in the Biacore software package. Antibodies were diluted to 10 ug/mL in the supplied acetate buffers to prescreen for the appropriate pH at which to conduct the immobilizations. For immobilizations, antibodies were diluted into the appropriate acetate buffer (10 mM acetate pH 4.0) and coupled directly to the chip surface using standard EDC chemistry at three different protein levels (500, 1000, and 1500 RU). The fourth flow cell was mock coupled with EDC to cap the carboxyl groups and provide a background surface as a negative control.

Binding Studies

Binding studies were performed by successive injections of varying concentrations of soluble human EPO receptor over the chip surface (500 RU immobilized protein). Binding analyses were performed in the supplied HBS-EP buffer [HBS buffer-10 mM HEPES pH=7.4, 150 mM NaCl, 3 mM EDTA, 0.005% Polysorbate 20 (v/v), Biacore] using receptor diluted to the desired concentrations (10-200 nM) using the running buffer (HBS-EP). Experiments were performed at a flow rate of 30 uL/min. The receptor was injected over a period of 3 minutes followed by a 15 minute dissociation period. Simultaneous injections over the flow cell created as a negative control were also performed. All injections were performed in triplicate.

Model Fitting

Data were fit to the models available in the BiaEvaluation 3.0.2 software package (Biacore). The data points from the experimental injections were corrected by subtraction of data points from simultaneous over the negative control surface. The corrected data were used to fit to the 1:1 (Langmuir) binding model as well as the bivalent analyte model available in the BiaEvaluation software package. Dissociation constants were calculated directly from fitting to the Langmuir binding model. For the bivalent analyte model, the dissociation constants were calculated indirectly using the calculated values for the kinetic dissociation and kinetic association constants, k_{d} and kₐ.

TABLE 6

| Antibody | kD |
|---|---|
| Ab 12 | 17.5 nM |
| Ab 198 | 13.9 nM |

### EXAMPLE 4

EPO Dependent Human Cell Proliferation Assay

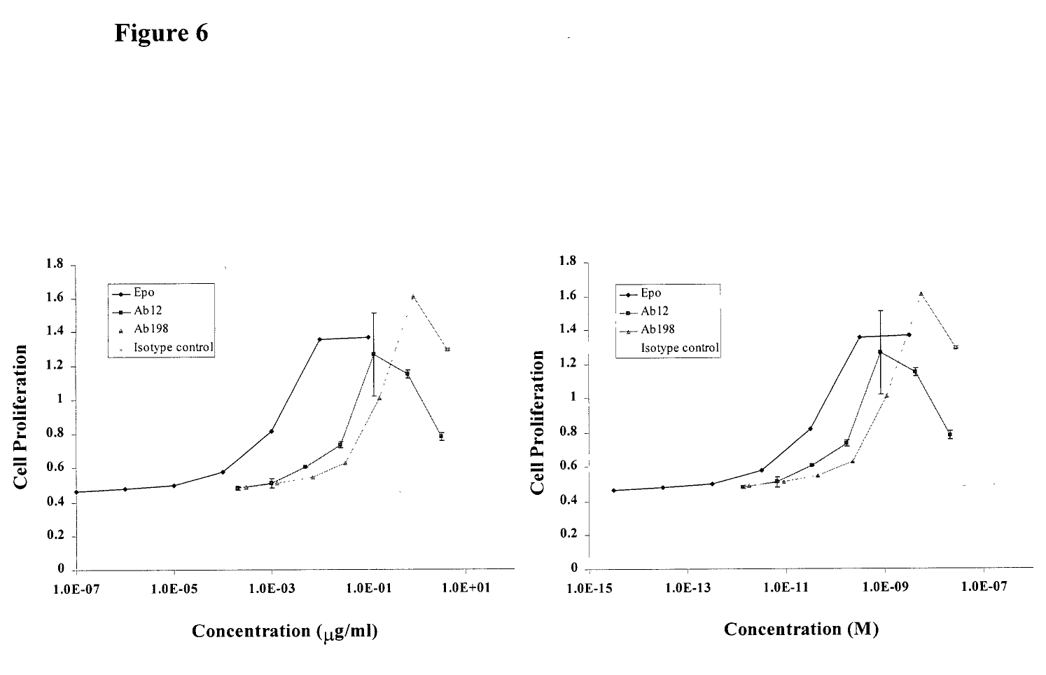

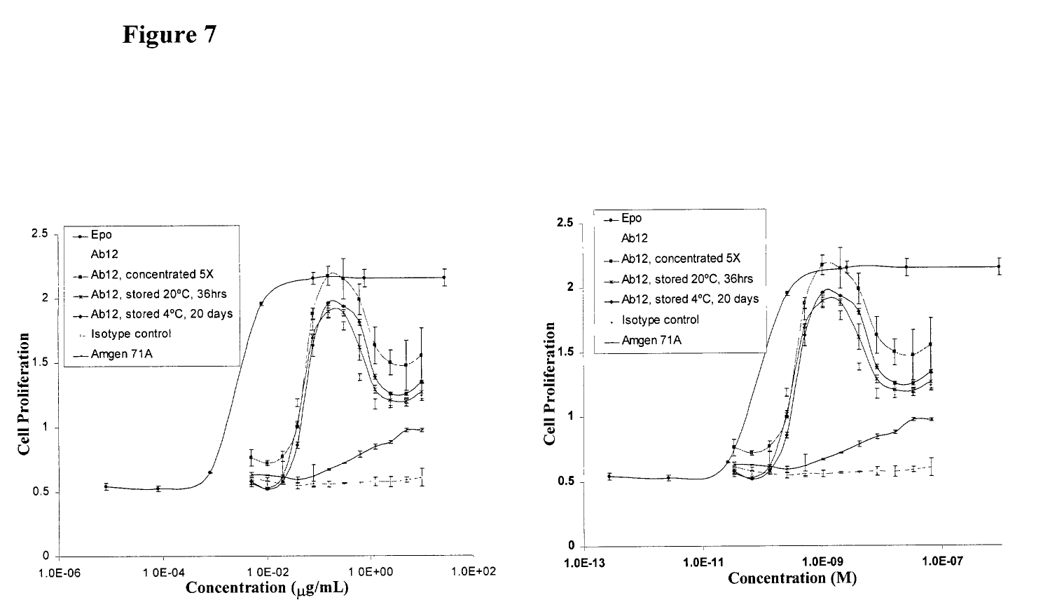

Stock cultures of the human erythroleukemic cell line, F36E cells were maintained in RPMI 1640 media with 10% fetal bovine serum and 1 unit per mL of recombinant human erythropoietin. Prior to assays, cells were cultured overnight at a density of 4.0 to 5.0×10⁵ cells per mL in growth medium without EPO. Cells were recovered, washed and resuspended at a density of 1.0×10⁶ cells per mL in assay medium (RPMI 1640+10% FBS) and 50 uL of cells added to wells of a 96 well microtiter plate. 50 uL of each of Ab12 and Ab198 or EPO standards (recombinant human EPO (rHuEPO)) in assay medium were added to wells and the plates were incubated in a humidified incubator at 37° C. with a 5% CO₂ atmosphere. After 72 hours, 20 μL of Promega Cell Titer 96 Aqueous® reagent (as prepared per manufacturer's instructions, Madison, Wis.) was added to all wells. Plates were incubated at 37° C. with a 5% CO₂ atmosphere for 4 hours and the optical density at 490 nm was determined using a microplate reader (Wallac Victor 1420 Multilabel Counter, Wallac Company, Boston, Mass.). The results are shown in FIG. 6. Purified Abs 12 and 198 stimulated proliferation of the F36E cell line. Maximal proliferative activity was similar to that observed with the EPO control and shown by a bell shaped curve as concentration increased. The results in FIG. 7 demonstrate that Ab12, after storage at 4° C. for up to 20 days, is active in inducing the proliferation of F36E cells. Proliferative activity was similar to that observed with the EPO control with the maximal response differing about ten-fold on a molar equivalent basis

### EXAMPLE 5

Human CD36+ CFUe Assay

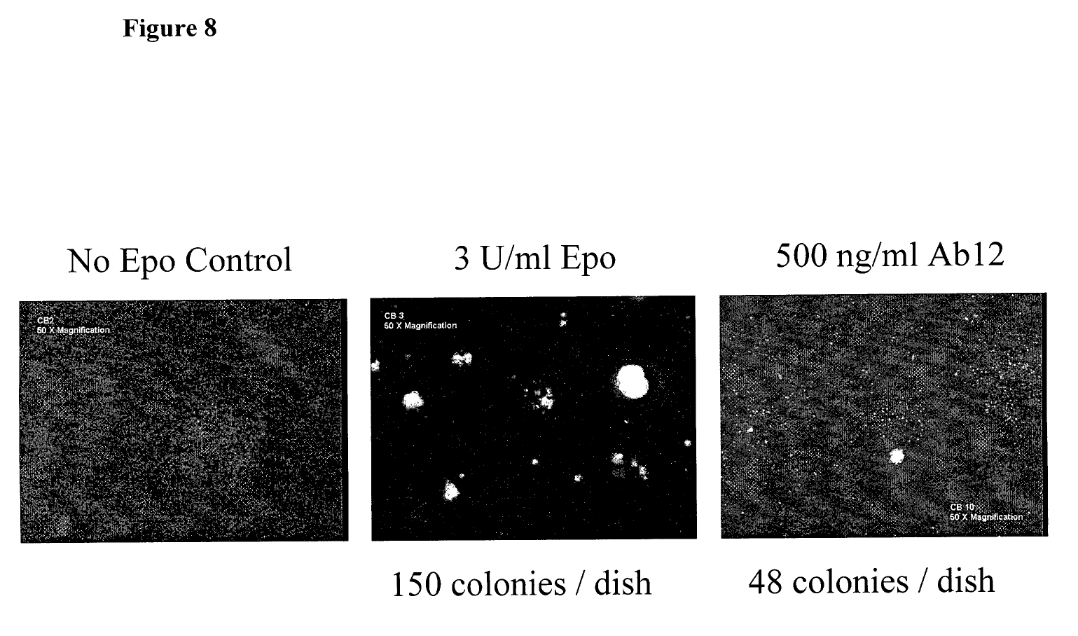

Frozen human CD36+ erythroid progenitor cells obtained from Poietics (Biowhittaker (Walkersville, Md.)) were thawed and at 10⁴ cells/ml in IMDM-2% FBS. Cells (0.3 ml) were added to 0.3 ml tubes containing 2.4 ml Methocult (StemCell Technologies, Vancouver, Canada) Cat. #04230), 0.3 ml stem cell growth factor (Sigma, St. Louis, Mo. Cat. #S7901, 100 ug/ml), and 0.3 ml EPO (R&D Systems), Ab 12, or IMDM-2% FBS. After mixing, 1.1 ml of the Methocult suspension was added to a 35 mm non tissue culture treated sterile petri dish and incubated at 37° C., 5% CO₂ for 2 weeks. Colonies were identified microscopically. The results are shown in FIG. 8. Specifically, Ab12 induced the formation of CFU-E colonies from human CD 36+ progenitor cells. The colonies, identified microscopically, were red in color. The size and number of the colonies is reduced compared to those observed with the EPO control probably due to a reduced proliferative signal.

### EXAMPLE 6

Demonstration of Erythopoietic Activity in Liquid Cultures

CD34+ cells were enriched from human peripheral blood using a Direct CD34+ Progenitor Cell Isolation Kit (Miltenyi, Auburn, Calif.). Recovered cells were washed twice with alpha-medium and re-suspended in suspension culture media (alpha-media supplemented with 30% FCS, 1% deionized BSA, 10⁻⁵M β-mercaptoethanol, 10⁻⁶ M dexamethasone, 0.3 mg/mL human hollo-transferrin and 10 ng/mL human recombinant stem cell factor). Cells were plated out at a density of 1×10⁴ cells/mL in duplicates in 6-well microplates with test antibody at concentrations ranging from 0.1-100 ng/mL. Plates were incubated at 37° C. and 5% CO₂ for two weeks. Duplicate samples from each well were recovered for cell counts and staining with benzidine (Reference Fibach, E., 1998 Hemoglobin, 22:5-6, 445-458).

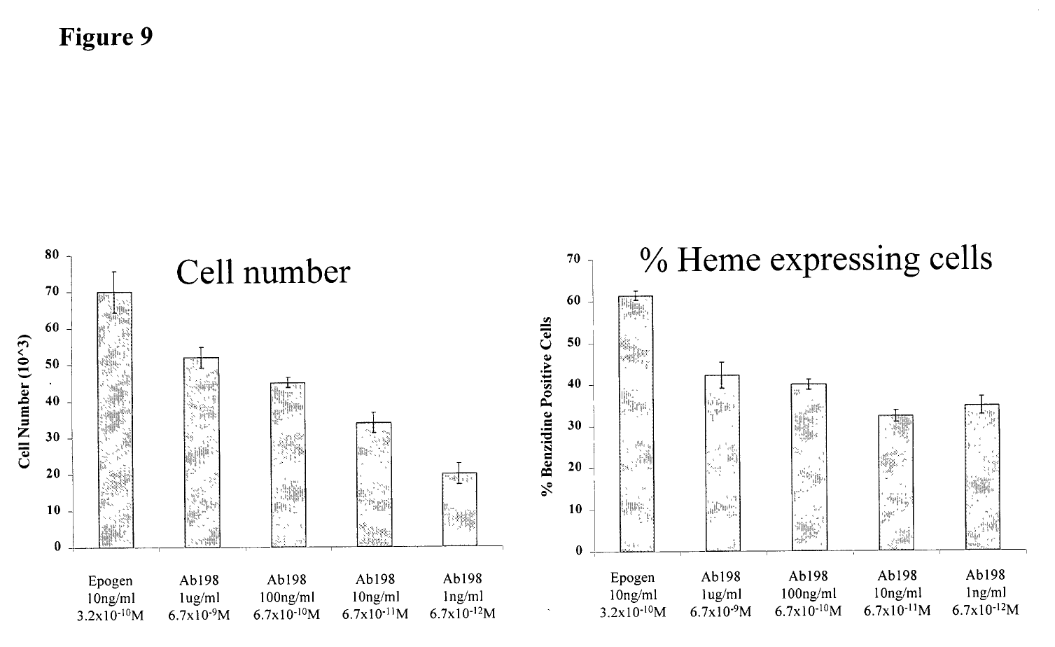

The results are shown in FIG. 9. Specifically, Ab198 induced the proliferation of human erythroid producing cells derived from progenitor cells in a dose dependent manner. The number of proliferating cells and the percentage expressing hemoglobin, as indicated by staining with benzidine, was reduced compared to the EPO treated controls again probably due to a reduced proliferative signal.

### EXAMPLE 7

Cynomolgus Bone Marrow CFUe Assay

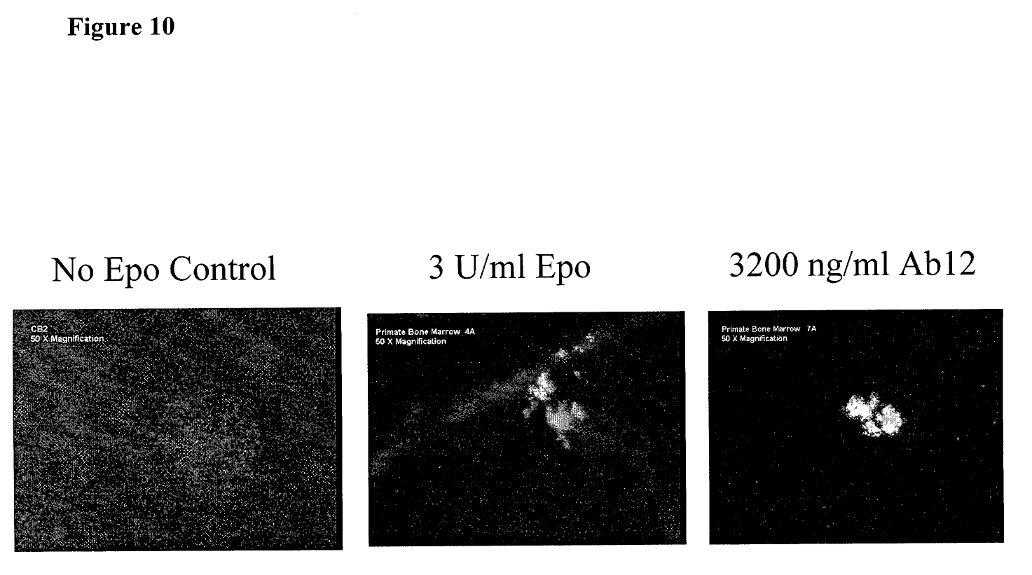

Bone marrow was harvested from cynomolgus monkeys and diluted 1:2 with PBS. Three ml of the diluted bone marrow was layered over six ml of Lymphoprep (Gibco (Invitrogen), Carlsbad, Calif. Cat. #1001967), centrifuged at 2700 rpm for 20 minutes and the buffy coat recovered and diluted in 10 ml IMDM-2% FBS. Cells were centrifuged and resuspended at 10⁶ cells/ml in IMDM-2% FBS. Cells (0.3 ml) were added to tubes containing 2.4 ml Methocult (StemCell Technologies, Vancouver, Canada) Cat. #04230), 0.3 ml stem cell growth factor (Sigma, Cat. #S7901, 100 ug/ml), 0.3 ml EPO (R& D Systems, Minneapolis, Minn.), test antibody (Ab198), or IMDM-2% FBS. After mixing, 1.1 ml of the Methocult suspension was added to a 35 mm non tissue culture treated sterile petri dish and incubated at 37° C., 5% CO₂ for 2 weeks. Colonies were identified microscopically. The results of this assay are shown in FIG. 10 demonstrate that Ab198 induced the formation of CFU-E colonies (although the number of colonies was reduced compared to that observed with the EPO control).

### EXAMPLE 8

ELISA to Measure Binding of SE-3 Peptide

96 well polystyrene plates (Dynatec (Elk Grove Village, Ill.) Immunolon 4) were coated with 80 ul of 5 ug/ml soluble EPO receptor (sEPOR) (R&D Systems (Minneapolis, Minn.) Cat. #307-ER/LF), or peptide SE-3 (PGNYSFSYQLEDEPWKLCRLHWAPTARGAV) (described in U.S. Pat. No. 6,319,499) diluted in 0.015M Na₂CO₃, 0.035M NaHCO₃, pH 9.4 for 2 hours at room temperature and overnight at 4° C. Plates were blocked for 30 minutes at room temperature with 100 ul of 5% BSA in PBS (Gibco (Invitrogen (Carlsbad, Calif.)) Cat.#10010). After removal of blocking solution, 50 ul of Ab12 at 5 ug/ml in PBS with 1% BSA was added to wells and plates were incubated at room temperature for 2 hours. Plates were washed three times using a Skatron 400 Plate Washer with PBS/0.05% Tween 20 and 50 ul of secondary antibody diluted in PBS/0.25% BSA/0.05% Tween 20 added to the wells. For Ab12, goat anti-human IgG (Fc)-HRP (Caltag (Burlingame, Calif.) Cat.#H10507) diluted 1:1000 was used and for Ab 71A (available from the American Type Culture Collection HB 11689, also described in U.S. Pat. No. 6,319,499), goat anti mouse IgG (Fc)-HRP (Jackson Laboratories (West Grove, Pa.) Cat.#115-035-164) diluted 1:5000 was used. After a 1 hour incubation at room temperature, plates were washed three times as before and 50 ul of OPD Developing Reagent (Sigma #P9187) added to each well. Color development was stopped by addition of 50 ul of 1N HCl to the wells and optical density measured at 490 nm on a Victor 1420 Multi-Label Counter.

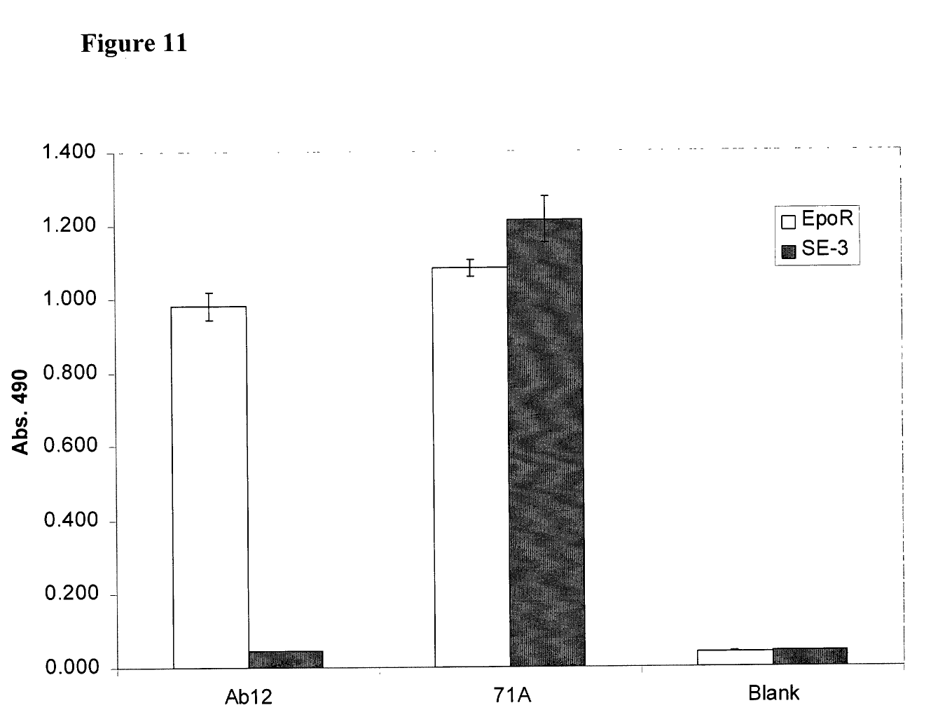

FIG. 11 shows that Abs 12 and 198 do not interact (i.e. bind) with SE-3 peptide. Ab 71 A does interact (i.e. binds) with the SE-3 peptide All three Abs (12, 198 and 71A) interacted with immobilized erythropoietin receptor.

### EXAMPLE 9

EPO Dependent Proliferation Assay

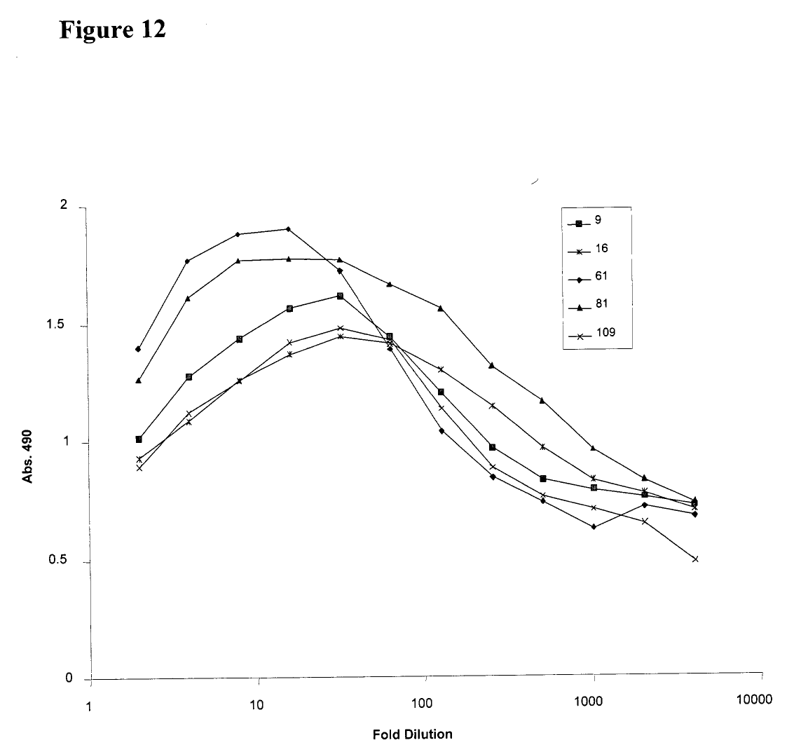

Primary hybridoma supernatants were diluted in assay medium and tested for their ability to stimulate the proliferation of the F36E human erythroleukemic cells as described in EXAMPLE 5. Results with five primary supernatants are shown in FIG. 12. These samples stimulated the proliferation of F36E cells with a more broad titration curve than observed with Abs 12 or 198 (see FIGS. 6 and 7)

### EXAMPLE 10

ELISA to Measure Binding of Hybridoma Supernatants to SE-3 Peptide

Forty-two primary hybridoma supernatants were tested for their ability to bind to either immobilized EPO receptor or peptide SE-3 as described in EXAMPLE 10. FIG. 13 shows that whereas all the hybridoma supernatants tested interact with immobilized EPO receptor, only sample 16 interacted with SE-3 peptide at levels above background.

All abstracts, references, patents and published patent applications referred to herein are hereby incorporated by reference.

The present invention is illustrated by way of the foregoing description and examples. The foregoing description is intended as a non-limiting illustration, since many variations will become apparent to those skilled in the art in view thereof.

Changes can be made to the composition, operation and arrangement of the method of the present invention described herein without departing from the concept and scope of the invention.

What is claimed is:

1. An antibody or antibody fragment thereof that activates an endogenous activity of a human erythropoietin receptor in a mammal but does not interact with a peptide having an amino acid sequence of PGNYSFSYQLEDEPWKLCRLHQAPTARGAV (SEQ ID NO:1).

2. An antibody or antibody fragment thereof that is capable of activating an endogenous activity of a human erythropoietin receptor in a mammal, wherein said antibody or antibody fragment thereof exhibits a binding affinity within one hundred fold of the binding affinity of endogenous human erythropoietin to the erythropoietin receptor.

3. An antibody or antibody fragment thereof that activates an endogenous activity of a human erythropoietin receptor in a mammal, comprising
at least one heavy chain variable region having the amino acid sequence of SEQ ID NO: 3 or antibody fragment thereof; wherein said antibody or antibody fragment thereof does not interact with a peptide having an amino acid sequence of PGNYSFSYQLEDEPWKLCRLHQAPTARGAV (SEQ ID NO:1).

4. An antibody or antibody fragment thereof that activates an endogenous activity of a human erythropoietin receptor in a mammal, comprising
at least one heavy chain variable region having the amino acid sequence of SEQ ID NO: 5 or antibody fragment thereof; wherein said antibody or antibody fragment thereof does not interact with a peptide having an amino acid sequence of PGNYSFSYQLEDEPWKLCRLHQAPTARGAV (SEQ ID NO:1).

5. An antibody or antibody fragment thereof that activates an endogenous activity of a human erythropoietin receptor in a mammal, comprising:
at least one heavy chain variable region having the amino acid sequence of SEQ ID NO: 3 or antibody fragment thereof; and
at least one light chain variable region having the amino acid sequence of SEQ ID NO: 5 or antibody fragment thereof,
wherein said antibody or antibody fragment thereof does not interact with a peptide having an amino acid sequence of PGNYSFSYQLEDEPWKLCRLHQAPTARGAV (SEQ ID NO:1).

6. An antibody or antibody fragment thereof that activates an endogenous activity of a human erythropoietin receptor in a mammal, comprising
at least one heavy chain variable region having the amino acid sequence of SEQ ID NO: 7 or antibody fragment thereof; wherein said antibody or antibody fragment thereof does not interact with a peptide having an amino acid sequence of PGNYSFSYQLEDEPWKLCRLHQAPTARGAV (SEQ ID NO:1).

7. An antibody or antibody fragment thereof that activates an endogenous activity of a human erythropoietin receptor in a mammal, comprising
at least one heavy chain variable region having the amino acid sequence of SEQ ID NO: 9 or antibody fragment thereof; wherein said antibody or antibody fragment thereof does not interact with a peptide having an amino acid sequence of PGNYSFSYQLEDEPWKLCRLHQAPTARGAV (SEQ ID NO:1).

8. An antibody or antibody fragment thereof that activates an endogenous activity of a human erythropoietin receptor in a mammal, said antibody comprising:
at least one heavy chain variable region having the amino acid sequence of SEQ ID NO: 7 or antibody fragment thereof; and
at least one light chain variable region having the amino acid sequence of SEQ ID NO: 9 or antibody fragment thereof,
wherein said antibody or antibody fragment thereof does not interact with a peptide having an amino acid sequence of PGNYSFSYQLEDEPWKLCRLHQAPTARGAV (SEQ ID NO:1).

9. An isolated antibody or antibody fragment thereof capable of binding to a human erythropoietin receptor in a mammal, said antibody comprising:
at least one heavy chain variable region having the amino acid sequence of SEQ ID NO: 3 or antibody fragment thereof.

10. An isolated antibody or antibody fragment thereof capable of binding to a human erythropoietin receptor in a mammal, said antibody comprising:
at least one light chain variable region having the amino acid sequence of SEQ ID NO: 5 or antibody fragment thereof.

11. An isolated antibody or antibody fragment thereof capable of binding to a human erythropoietin receptor in a mammal, said antibody comprising:
at least one heavy chain variable region having the amino acid sequence of SEQ ID NO: 7 or antibody fragment thereof.

12. An isolated antibody or antibody fragment thereof capable of binding to a human erythropoietin receptor in a mammal, said antibody comprising:
at least one light chain variable region having the amino acid sequence of SEQ ID NO: 9 or antibody fragment thereof.

13. An isolated antibody or antibody fragment thereof capable of binding to a human erythropoietin receptor in a mammal, said antibody comprising:
at least one heavy chain variable region having the amino acid sequence of SEQ ID NO: 11 or antibody fragment thereof.

14. An isolated antibody or antibody fragment thereof capable of binding to a human erythropoietin receptor in a mammal, said antibody comprising:
at least one light chain variable region having the amino acid sequence of SEQ ID NO: 13 or antibody fragment thereof.

15. An isolated antibody or antibody fragment thereof capable of binding to a human erythropoietin receptor in a mammal, said antibody comprising:
at least one heavy chain variable region having the amino acid sequence of SEQ ID NO: 15 or antibody fragment thereof.

16. An isolated antibody or antibody fragment thereof capable of binding to a human erythropoietin receptor in a mammal, said antibody comprising:
at least one light chain variable region having the amino acid sequence of SEQ ID NO: 17 or antibody fragment thereof.

17. An isolated antibody or antibody fragment thereof capable of binding to a human erythropoietin receptor in a mammal, said antibody comprising:
at least one heavy chain variable region having the amino acid sequence of SEQ ID NO: 19 or antibody fragment thereof.

18. An isolated antibody or antibody fragment thereof capable of binding to a human erythropoietin receptor in a mammal, said antibody comprising:
at least one light chain variable region having the amino acid sequence of SEQ ID NO: 21 or antibody fragment thereof.

19. An isolated antibody or antibody fragment thereof capable of binding to a human erythropoietin receptor in a mammal, said antibody comprising:
at least one heavy chain variable region having the amino acid sequence of SEQ ID NO: 23 or antibody fragment thereof.

20. An isolated antibody or antibody fragment thereof capable of binding to a human erythropoietin receptor in a mammal, said antibody comprising:
at least one light chain variable region having the amino acid sequence of SEQ ID NO: 25 or antibody fragment thereof.

21. An isolated antibody or antibody fragment thereof capable of binding to a human erythropoietin receptor in a mammal, said antibody comprising:
at least one heavy chain variable region having the amino acid sequence of SEQ ID NO:27 or antibody fragment thereof.

22. An isolated antibody or antibody fragment thereof capable of binding to a human erythropoietin receptor in a mammal, said antibody comprising:
at least one light chain variable region having the amino acid sequence of SEQ ID NO:29 or antibody fragment thereof.

23. An isolated antibody or antibody fragment thereof capable of binding to a human erythropoietin receptor in a mammal, said antibody comprising:
at least one heavy chain variable region having the amino acid sequence of SEQ ID NO:31 or antibody fragment thereof.

24. An isolated antibody or antibody fragment thereof capable of binding to a human erythropoietin receptor in a mammal, said antibody comprising:
at least one light chain variable region having the amino acid sequence of SEQ ID NO:33 or antibody fragment thereof.

25. An isolated antibody or antibody fragment thereof capable of binding to a human erythropoietin receptor in a mammal, said antibody comprising:
at least one heavy chain variable region having the amino acid sequence of SEQ ID NO: 35 or antibody fragment thereof.

26. An isolated antibody or antibody fragment thereof capable of binding to a human erythropoietin receptor in a mammal, said antibody comprising:
at least one light chain variable region having the amino acid sequence of SEQ ID NO: 37 or antibody fragment thereof.

27. An isolated antibody or antibody fragment thereof capable of binding to a human erythropoietin receptor in a mammal, said antibody comprising:
at least one heavy chain variable region having the amino acid sequence of SEQ ID NO: 39 or antibody fragment thereof.

28. An isolated antibody or antibody fragment thereof capable of binding to a human erythropoietin receptor in a mammal, said antibody comprising:
at least one light chain variable region having the amino acid sequence of SEQ ID NO:41 or antibody fragment thereof.

29. An isolated antibody or antibody fragment thereof capable of binding to a human erythropoietin receptor in a mammal, said antibody comprising:
at least one heavy chain variable region having the amino acid sequence of SEQ ID NO: 43 or antibody fragment thereof.

30. An isolated antibody or antibody fragment thereof capable of binding to a human erythropoietin receptor in a mammal, said antibody comprising:
at least one light chain variable region having the amino acid sequence of SEQ ID NO: 45 or antibody fragment thereof.

31. An isolated antibody capable of binding a human erythropoietin receptor in a mammal, said antibody comprising a heavy chain variable region comprising a continuous sequence from CDR1 through CDR3 having the amino acid sequence selected from the group consisting of: SEQ ID NO:46, SEQ ID NO:47, SEQ ID NO:48, SEQ ID NO:49, SEQ ID NO:50 and fragments thereof.

32. An isolated antibody capable of binding a human erythropoietin receptor in a mammal, said antibody comprising a light chain variable region comprising a continuous sequence from CDR1 through CDR3 having the amino acid sequence selected from the group consisting of: SEQ ID NO:51, SEQ ID NO:52, SEQ ID NO:53, SEQ ID NO:54, SEQ ID NO:55, SEQ ID NO:56, SEQ ID NO:57 and fragments thereof.

33. A method of activating an endogenous activity of a human erythropoietin receptor in a mammal, the method comprising the step of administering to said mammal a therapeutically effective amount of an antibody or antibody fragment thereof to activate said receptor, wherein said antibody or antibody fragment thereof does not interact with a peptide having an amino acid sequence of PGNYSFSYQLEDEPWKLCRLHQAPTARGAV (SEQ ID NO:1).

34. A method of modulating an endogenous activity of a human erythropoietin receptor in a mammal, the method comprising the step of administering to a mammal a therapeutically effective amount of the antibody or antibody fragment of claim 1 to modulate the activity of the receptor.

35. A method of treating a mammal suffering aplasia, the method comprising the step of administering to a mammal in need of treatment a therapeutically effective amount of an antibody or antibody fragment thereof to activate said receptor, wherein said antibody or antibody fragment thereof does not interact with a peptide having an amino acid sequence of PGNYSFSYQLEDEPWKLCRLHQAPTARGAV (SEQ ID NO:1).

36. A method of treating a mammal suffering aplasia, the method comprising the step of administering to a mammal in need of treatment a therapeutically effective amount of the antibody or antibody fragment of claim 1 to modulate the activity of the receptor.

37. A pharmaceutical composition comprising a therapeutically effective amount of a pharmaceutically acceptable excipient and an antibody or antibody fragment thereof, wherein said antibody or antibody fragment thereof does not interact with a peptide having an amino acid sequence of: PGNYSFSYQLEDEPWKLCRLHQAPTARGAV (SEQ ID NO:1).

38. An isolated and purified polynucleotide sequence selected from the group consisting of: SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42 and SEQ ID NO:44, fragments, complements, and degenerate codon equivalents thereof.

39. An isolated and purified amino acid sequence selected from the group consisting of: SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57 and fragments thereof.
